(19) United States
(12) Patent Application Publication
(10) Pub. No.: US 2025/0290149 A1
(43) Pub. Date: Sep. 18, 2025

(54) SYSTEMS AND METHODS FOR ENRICHING CELL-FREE MICROBIAL NUCLEIC ACID MOLECULES

(71) Applicant: Liquid Biopsy Holdco, LLC, Bentonville, AR (US)

(72) Inventors: Eddie ADAMS, San Diego, CA (US); Serena FRARACCIO, San Diego, CA (US)

(21) Appl. No.: 18/862,257

(22) PCT Filed: May 2, 2023
(86) PCT No.: PCT/US2023/066519

§ 371 Date: Nov. 1, 2024

Related U.S. Application Data
(60) Provisional application No. 63/337,889, filed on May 3, 2022.

(51) Int. Cl. C12Q 1/6886; C12Q 1/6806; C12Q 1/689; C12Q 1/6895; G16B 40/00; G16H 50/20

(52) U.S. Cl. CPC C12Q 1/6886; C12Q 1/6806; C12Q 1/689; C12Q 1/6895; G16B 40/00; G16H 50/20; C12Q 2600/156

(57) ABSTRACT

Provided are systems and methods for enriching cell-free microbial nucleic acids from one or more subjects' samples.

### CROSS REFERENCE

This application claims the benefit of U.S. Provisional Application No. 63/337,889 filed May 3, 2022, which is hereby incorporated by reference in its entirety for all purposes.

### BACKGROUND

Circulating microbial cell-free nucleic acid molecules have shown promise as disease diagnostic and/or prognostic biomarkers. However, the relevant microbial cell-free nucleic acid signatures are often present in small quantities and are challenging to detect amongst a background of mammalian cell-free nucleic acids. Therefore, there exists an unmet need for systems and methods to improve the detection of circulating microbial cell-free nucleic acid molecules used for disease diagnosis and/or prognosis.

### SUMMARY

The current invention addresses the unmet need with methods and systems configured to deplete mammalian cell-free nucleic acids from a sample, thereby enriching and/or isolating microbial cell-free nucleic acids. In some cases, the mammalian cell-free nucleic acids may be depleted through affinity agents, configured to selectively bind to one or more mammalian cell-free nucleic acid molecules. In some instances, the one or more mammalian cell-free nucleic acid molecules may be complexed and/or coupled to one or more proteins. In some cases, the one or more proteins comprise histone proteins.

Aspects of the disclosure comprise a method of generating a microbial metagenomic feature set for differentiating a cancer and non-oncologic disease of one or more subjects. In some embodiments, the method comprises: (a) providing a biological sample of one or more subjects comprising one or more mammalian nucleic acid molecules and one or more microbial nucleic acid molecules and corresponding health states; (b) removing said one or more mammalian nucleic acid molecules from said biological sample with one or more affinity capture reagents; (c) sequencing the remaining one or more microbial nucleic acid molecules to generate one or more microbial sequencing reads; and (d) generating a microbial metagenomic feature set configured to differentiate a presence of cancer or non-cancer disease by combining one or more metagenomic feature abundances of said one or more microbial sequencing reads and said health states of said one or more subjects. In some embodiments, step (b) comprises: (a) contacting said liquid biological sample with a solid support comprising immobilized anti-nucleosome antibodies to form antibody-nucleosome interaction complexes; (b) separating said solid support from said liquid biological sample to concentrate said antibody-nucleosome interaction complexes; and (c) purifying the remaining one or more nucleosome-depleted microbial nucleic acid molecules. In some embodiments, the anti-nucleosome antibodies are configured to bind to an epitope comprising DNA and one or more histone proteins. In some embodiments, the solid supports comprise a magnetic bead, agarose bead, non-magnetic latex, functionalized Sepharose, pH-sensitive polymers or any combination thereof.

In some embodiments, the metagenomic feature set comprises microbial taxonomic abundance. In some embodiments, the metagenomic feature set comprises computationally inferred microbial biochemical pathways and said microbial biochemical pathways' associated abundances. In some embodiments, the metagenomic feature set comprises microbial phylogenetic marker genes or marker gene fragments thereof.

In some embodiments, step (b) comprises: (a) contacting said liquid biological sample with one or more anti-nucleosome antibodies to form antibody-nucleosome interaction complexes; (b) contacting said antibody-nucleosome interaction complexes with a solid support, wherein a surface of said solid support comprises a binding moiety configured to couple to said antibody-nucleosome interaction complex; (c) separating said solid support from said liquid biological sample to concentrate said antibody-nucleosome interaction complexes; and (d) purifying the remaining one or more nucleosome-depleted microbial nucleic acid molecules. In some embodiments, the one or more anti-nucleosome antibodies comprise one or more epitope tags. In some embodiments, the one or more epitope tags comprise an N- or C-terminal 6×-histidine tag, green fluorescent protein (GFP), myc, hemagglutinin (HA), Fc fusion, biotin or any combination thereof. In some embodiments, the solid supports comprise a magnetic bead, agarose bead, non-magnetic latex, functionalized Sepharose, pH-sensitive polymers or any combination thereof. In some embodiments, the solid support comprises covalently immobilized affinity agents. In some embodiments, the affinity reagents comprise streptavidin, antibodies specific for 6×-histidine tag, green fluorescent protein (GFP), myc, hemagglutinin (HA), biotin, or any combination thereof. In some embodiments, the affinity agents comprise anti-species antibodies.

In some embodiments, step (c) comprises: (a) generating single-stranded DNA libraries from the one or more microbial nucleic acid molecules; (b) performing shotgun metagenomic sequencing analysis of said single-stranded DNA libraries to produce one or more sequencing reads; (c) filtering said one or more sequencing reads to produce one or more mammalian DNA-depleted microbial sequencing reads; and (d) decontaminating said one or more mammalian DNA-depleted microbial sequencing reads to remove non-endogenous microbial sequencing reads. In some embodiments, the decontaminating comprises in-silico decontamination. In some embodiments, the filtering comprises computationally mapping said one or more sequencing reads to a human reference genome database.

In some embodiments, the biological sample comprises a liquid biological, where the liquid biological sample comprises: plasma, serum, whole blood, urine, cerebral spinal fluid, saliva, sweat, tears, exhaled breath condensate, or any dilution, or processed fraction thereof.

In some embodiments, the step (c) comprises: (a) amplifying one or more genomic features of said one or more microbial nucleic acid molecules, thereby generating an amplified one or more genomic features; (b) sequencing said amplified one or more genomic features to generate one or more sequencing reads; (c) filtering said one or more sequencing reads to produce one or more mitochondrial DNA-depleted microbial sequencing reads; and (d) decontaminating said one or more mitochondrial DNA-depleted microbial sequencing reads to remove non-endogenous microbial sequencing reads. In some embodiments, the decontaminating comprises in-silico decontamination. In some embodiments, the one or more genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof.

In some embodiments, the said bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) comprises enriching said one or more microbial nucleic acid molecules. In some embodiments, enriching comprises: (a) combining purified nucleosome-depleted microbial nucleic acid molecules with hybridization probes, wherein said hybridization probes comprise a nucleic acid sequence complementarity to microbial genomic features; (b) incubating said hybridization probes and said one or more nucleosome-depleted microbial nucleic acid molecules under conditions that promote nucleic acid base pairing between target nucleic acid features and said hybridization probes; (c) separating unbound hybridization probes and hybridized probes bound to said microbial nucleic acid molecules; and (d) washing said hybridized probes bound to said microbial nucleic acid molecules, thereby generating one or more enriched microbial nucleic acid molecules. In some embodiments, washing is to remove non-specifically associated nucleic acid molecules and other reaction components.

In some embodiments, enriching comprises: (a) combining one or more purified nucleosome-depleted microbial nucleic acid molecules with one or more recombinant CXXC-domain proteins to form a protein-DNA binding reaction; (b) incubating said protein-DNA binding reaction under conditions that promote an interaction between said recombinant CXXC-domain proteins and non-methylated CpG motifs of said one or more nucleosome-depleted microbial nucleic acid molecules; (c) separating unbound recombinant CXXC-domain proteins and recombinant CXXC-domain proteins bound to said non-methylated CpG motifs from a remainder of said protein-DNA binding reaction; (d) washing said recombinant CXXC-domain proteins bound to said non-methylated CpG nucleic acid fragments, thereby generating one or more enriched nucleic acid molecules for amplification. In some embodiments, the washing is configured to remove non-specifically associated nucleic acid molecules and said remainder of protein-DNA binding reaction components. In some embodiments, the amplification comprises performing a polymerase chain reaction or derivatives thereof. In some embodiments, the derivatives thereof comprise inverse PCR, anchored PCR, primer-directed rolling circle amplification, or any combination thereof.

In some embodiments, the one or more subjects comprise human, non-human mammal, or any combination thereof subjects. In some embodiments, the one or more mammalian nucleic acid molecules comprise DNA, RNA, cell-free DNA, cell-free RNA, exosomal DNA, exosomal RNA, or any combination thereof nucleic acid molecules, and wherein said one or more microbial nucleic acid molecules comprise microbial cell-free RNA, microbial cell-free DNA, microbial RNA, microbial DNA, or any combination thereof nucleic acid molecules. In some embodiments, the said cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the non-oncologic disease comprises healthy, disease, or any combination thereof non-cancer state. In some embodiments, the disease state comprises benign neoplasms of the integumentary, skeletal, muscular, nervous, endocrine, cardiovascular, lymphatic, digestive, respiratory, urinary, reproductive, or any system combinations thereof. In some embodiments, the cancer comprises a cancer of stage I, II, or III. In some embodiments, the method further comprises generating a trained predictive model, wherein said trained predictive model is trained with said microbial metagenomic feature set and said health state of said one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, said subject's or subjects' health states comprise said subjects' known non-oncologic disease, cancer, or any combination thereof.

Another aspects of the disclosure comprises a method of using an output of a trained predictive model to diagnose a cancer or non-oncologic disease of one or more subjects, the method comprising: (a) providing a biological sample of one or more subjects comprising one or more mammalian nucleic acid molecules and one or more microbial nucleic acid molecules; (b) removing said one or more mammalian nucleic acid molecules from said biological sample with one or more affinity capture reagents; (c) sequencing the remaining one or more microbial nucleic acid molecules to generate one or more microbial sequencing reads; (d) generating one or more microbial metagenomic feature sets by combining one or more metagenomic feature abundances of said one or more microbial sequencing reads; and (e) outputting a diagnosis of a cancer or non-oncologic disease of said one or more subjects at least as a result of providing said one or more microbial metagenomic feature sets as an input to a trained predictive model. In some embodiments, the one or more microbial metagenomic feature sets comprise microbial taxonomic abundance. In some embodiments, the one or more microbial metagenomic feature sets comprise computationally inferred microbial biochemical pathways and their associated abundance. In some embodiments, the one or more microbial metagenomic feature sets comprise microbial phylogenetic marker genes or marker gene fragments thereof.

In some embodiments, the biological sample comprises a liquid biological sample, where the liquid biological sample comprises plasma, serum, whole blood, urine, cerebral spinal fluid, saliva, sweat, tears, exhaled breath condensate, or any combination, dilution, or processed fraction thereof.

In some embodiments, step (b) comprises: (a) contacting said liquid biological sample with a solid support comprising immobilized anti-nucleosome antibodies, wherein said anti-nucleosome antibodies are configured to form antibody-nucleosome interaction complexes; (b) separating said solid support from said liquid biological sample to concentrate said antibody-nucleosome interaction complexes; and (c) purifying the remaining one or more nucleosome-depleted microbial nucleic acid molecules. In some embodiments, the anti-nucleosome antibodies recognize an epitope comprising DNA and one or more histone proteins. In some embodiments, the solid supports may comprise a magnetic bead, an agarose bead, non-magnetic latex, functionalized Sepharose, pH-sensitive polymers or any combinations thereof.

In some embodiments, step (b) comprises: (a) contacting said liquid biological sample with one or more anti-nucleosome antibodies to form antibody-nucleosome interaction complexes; (b) contacting said antibody-nucleosome interaction complexes with a solid support configured to bind to said antibody-nucleosome interaction complexes; (c) separating said solid support from said liquid biological sample to concentrate said antibody-nucleosome interaction complexes; and (d) purifying the remaining one or more nucleosome-depleted microbial nucleic acids. In some embodiments, the one or more anti-nucleosome antibodies comprise one or more epitope tags. In some embodiments, the one or more epitope tags comprise an N- or C-terminal 6×-histidine tag, green fluorescent protein (GFP), myc, hemagglutinin (HA), Fc fusion, biotin or any combination thereof. In some embodiments, the solid supports comprise a magnetic bead, agarose bead, non-magnetic latex, functionalized Sepharose, pH-sensitive polymers or any combination thereof. In some embodiments, the solid support comprises covalently immobilized affinity agents. In some embodiments, the covalently immobilized affinity agents comprise streptavidin, antibodies specific for 6×-histidine tag, green fluorescent protein (GFP), myc, hemagglutinin (HA), biotin, or any combination thereof. In some embodiments, the covalently immobilized affinity agents comprise anti-species antibodies.

In some embodiments, step (c) comprises: (a) generating single-stranded DNA libraries from said one or more microbial nucleic acid molecules; (b) performing shotgun metagenomic sequencing analysis of said single-stranded DNA libraries to produce one or more sequencing reads; (c) filtering said one or more sequencing reads to produce one or more mammalian DNA-depleted microbial sequencing reads; and (d) decontaminating said one or more mammalian DNA-depleted microbial sequencing reads to remove non-endogenous microbial sequencing reads. In some embodiments, decontaminating comprises in-silico decontamination of said one or more mammalian DNA-depleted microbial sequencing reads. In some embodiments, filtering comprises computationally mapping said one or more sequencing reads to a human reference genome database.

In some embodiments, step (c) comprises: (a) amplifying one or more genomic features of said one or more microbial nucleic acid molecules, thereby generating an amplified one or more genomic features; (b) sequencing said amplified one or more genomic features to generate one or more sequencing reads; (c) filtering said one or more sequencing reads to produce one or more mitochondrial DNA-depleted microbial sequencing reads; and (d) decontaminating said one or more mitochondrial DNA-depleted microbial sequencing reads to remove non-endogenous microbial sequencing reads. In some embodiments, decontaminating comprises in-silico decontamination of said one or more mitochondrial DNA-depleted microbial sequencing reads. In some embodiments, the one or more genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) comprises enriching said one or more microbial nucleic acid molecules. In some embodiments, enriching of said one or more microbial nucleic acid molecules comprises: (a) combining purified one or more nucleosome-depleted microbial nucleic acid molecules with hybridization probes, wherein said hybridization probes comprise a nucleic acid sequence complimentary to microbial genomic nucleic acid features; (b) incubating said hybridization probes and said one or more nucleosome-depleted microbial nucleic acid molecules under conditions that promote nucleic acid base pairing between said microbial genomic nucleic acid features and said hybridization probes; (c) separating unbound hybridization probes and hybridized probes bound to said one or more nucleosome-depleted microbial nucleic acid molecules; and (d) washing said hybridized probes bound to said one or more nucleosome-depleted microbial nucleic acid molecules, thereby generating one or more enriched microbial nucleic acid molecules. In some embodiments, the washing is configured to remove non-specifically associated nucleic acid molecules and other reaction components

In some embodiments, enriching said one or more microbial nucleic acid molecules comprises: (a) combining one or more purified nucleosome-depleted microbial nucleic acid molecules with one or more recombinant CXXC-domain proteins to form a protein-DNA binding reaction; (b) incubating said protein-DNA binding reaction under conditions that promote an interaction between said recombinant CXXC-domain proteins and non-methylated CpG motifs of said one or more nucleosome-depleted microbial nucleic acid molecules; (c) separating unbound recombinant CXXC-domain proteins and recombinant CXXC-domain proteins bound to said non-methylated CpG nucleic acid fragments from a remainder of the protein-DNA binding reaction components; (d) washing said recombinant CXXC-domain proteins bound to said non-methylated CpG nucleic acid fragments, thereby generating one or more enriched nucleic acid molecules for amplification. In some embodiments, the washing is configured to remove non-specifically associated nucleic acid molecules and said remainder of said protein-DNA binding reaction components. In some embodiments, the amplification comprises performing a polymerase chain reaction or derivatives thereof. In some embodiments, the derivatives thereof comprise inverse PCR, anchored PCR, primer-directed rolling circle amplification, or any combination thereof. In some embodiments, the one or more mammalian nucleic acid molecules and said one or more microbial nucleic acid molecules are derived from one or more liquid biological samples of said one or more subjects. In some embodiments, the one or more subjects comprise human, non-human mammal, or any combination thereof subjects. In some embodiments, the one or more mammalian nucleic acid molecules comprise DNA, RNA, cell-free RNA, cell-free DNA, exosomal DNA, exosomal RNA, or any combination thereof nucleic acid molecules, and wherein said one or more microbial nucleic acid molecules comprise microbial cell-free DNA, microbial cell-free RNA, microbial DNA, microbial RNA, or any combination thereof nucleic acid molecules. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the said non-oncologic disease comprises healthy, disease, or any combination thereof non-cancer state. In some embodiments, the disease state comprises benign neoplasms of the integumentary, skeletal, muscular, nervous, endocrine, cardiovascular, lymphatic, digestive, respiratory, urinary, reproductive, or any system combinations thereof. In some embodiments, the cancer comprises a cancer of stage I, II, or III.

In some embodiments, the trained predictive model is trained with one or more microbial metagenomic feature sets and a corresponding health state of one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, said subject's or subjects' health states comprise said subjects' known non-oncologic disease, cancer, or any combination thereof.

Another aspect of the disclosure comprises a system for diagnosing a cancerous or non-cancerous health state of one or more subjects. In some embodiments, the system comprises: (a) a processor; and (b) a non-transitory computer readable storage medium including software configured to cause said processor to: (i) receive a subject's one or more mammalian nucleosome-depleted nucleic acid molecules' sequencing reads of said one or more subjects' liquid biological samples, wherein said one or more nucleic acid sequencing reads comprise one or more metagenomic features of one or more microbial nucleic acid molecules; and (ii) output a diagnosis of a cancerous or non-cancerous health state of said one or more subjects at least as a result of providing said one or more microbial nucleic acid sequencing reads' one or more metagenomic features as an input to a trained predictive model. In some embodiments, the one or more metagenomic features comprise microbial taxonomic abundance. In some embodiments, the one or more metagenomic features comprise computationally inferred microbial biochemical pathways and their associated abundance. In some embodiments, the one or more metagenomic features comprise microbial phylogenetic marker genes or marker gene fragments thereof. In some embodiments, the mammalian nucleosome-depleted nucleic acid molecule sequencing reads are obtained and/or received from the subjects' liquid biological samples, where the liquid biological sample comprise: plasma, serum, whole blood, urine, cerebral spinal fluid, saliva, sweat, tears, exhaled breath condensate, or any combination, dilution, or processed fraction thereof.

In some embodiments, the one or more mammalian nucleosome-depleted nucleic acid molecules' sequencing reads are produced by: (a) contacting said liquid biological sample with a solid support to form antibody-nucleosome interaction complexes, wherein said solid support comprises a surface comprising anti-nucleosome antibodies coupled thereto; (b) separating said solid support from said liquid biological sample to concentrate said antibody-nucleosome interaction complexes; (c) purifying said remaining one or more nucleosome-depleted microbial nucleic acid molecules; and (d) sequencing said purified one or more nucleosome-depleted microbial nucleic acid molecules. In some embodiments, the anti-nucleosome antibodies are configured to recognize an epitope comprising DNA and one or more histone proteins. In some embodiments, the solid supports comprise a magnetic bead, an agarose bead, non-magnetic latex, functionalized Sepharose, pH-sensitive polymers or any combinations thereof.

In some embodiments, the one or more mammalian nucleosome-depleted nucleic acid molecules' sequencing reads are produced by: (a) contacting said liquid biological sample with one or more anti-nucleosome antibodies to form antibody-nucleosome interaction complexes; (b) contacting said antibody-nucleosome interaction complexes with a solid support; (c) separating said solid support from said liquid biological sample to concentrate said antibody-nucleosome interaction complexes; (d) purifying the remaining one or more nucleosome-depleted microbial nucleic acid molecules; and (e) sequencing said purified one or more nucleosome-depleted microbial nucleic acid molecules. In some embodiments, the one or more anti-nucleosome antibodies comprise one or more epitope tags. In some embodiments, the one or more epitope tags comprises an N- or C-terminal 6×-histidine tag, green fluorescent protein (GFP), myc, hemagglutinin (HA), Fc fusion, biotin or any combination thereof. In some embodiments, the solid supports comprise a magnetic bead, agarose bead, non-magnetic latex, functionalized Sepharose, pH-sensitive polymers or any combination thereof. In some embodiments, the solid support comprises covalently immobilized affinity agents. In some embodiments, the covalently immobilized affinity agents comprise streptavidin, antibodies specific for 6×-histidine tag, green fluorescent protein (GFP), myc, hemagglutinin (HA), biotin, or any combination thereof. In some embodiments, the covalently immobilized affinity agents comprise anti-species antibodies.

In some embodiments, the one or more mammalian nucleosome-depleted nucleic acid molecules' sequencing reads are produced by: (a) generating single-stranded DNA libraries from said one or more microbial nucleic acid molecules; (b) performing shotgun metagenomic sequencing analysis of said single-stranded DNA libraries to produce one or more sequencing reads; (c) filtering said one or more sequencing reads to produce one or more mammalian DNA-depleted microbial sequencing reads; and (d) decontaminating said one or more mammalian DNA-depleted microbial sequencing reads to remove non-endogenous microbial sequencing reads. In some embodiments, the decontaminating comprises in-silico decontamination of said one or more mammalian DNA-depleted microbial sequencing reads. In some embodiments, filtering comprises computationally mapping said one or more sequencing reads to a human reference genome database.

In some embodiments, the mammalian nucleosome-depleted nucleic acid molecules' sequencing reads are produced by: (a) amplifying one or more genomic features of said one or more microbial nucleic acid molecules, thereby generating an amplified one or more genomic features; (b) sequencing said amplified one or more genomic features to generate one or more sequencing reads; (c) filtering said one or more sequencing reads to produce one or more mitochondrial DNA-depleted microbial sequencing reads; and (d) decontaminating said one or more mitochondrial DNA-depleted microbial sequencing reads to remove non-endogenous microbial sequencing reads. In some embodiments, the decontaminating comprises in-silico decontamination of said one or more mitochondrial DNA-depleted microbial sequencing reads. In some embodiments, the one or more genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, the amplifying comprises performing a polymerase chain reaction (PCR) or derivatives thereof. In some embodiments, the derivatives thereof comprise inverse PCR, anchored PCR, primer-directed rolling circle amplification, or any combination thereof. In some embodiments, the said one or more microbial nucleic acid molecules are enriched from said one or more mammalian nucleosome-depleted nucleic acid molecules.

In some embodiments, enriching of said one or more microbial nucleic acid molecules comprises: (a) combining purified nucleosome-depleted microbial nucleic acid molecules with hybridization probes, wherein said hybridization probes comprise a nucleic acid sequence complementarity to one or more microbial genomic nucleic acid features; (b) incubating said hybridization probes and one or more nucleosome-depleted microbial nucleic acid molecules under conditions that promote nucleic acid base pairing between said one or more microbial genomic nucleic acid features and said hybridization probes; (c) separating unbound hybridization probes and hybridized probes bound to said one or more nucleosome-depleted microbial nucleic acid molecules; and (d) washing said hybridized probes bound to said one or more nucleosome-depleted microbial nucleic acid molecules, thereby generating one or more enriched microbial nucleic acid molecules. In some embodiments, washing is configured to remove non-specifically associated nucleic acid molecules and other reaction components.

In some embodiments, enriching of said one or more microbial nucleic acid molecules comprises: (a) combining one or more purified nucleosome-depleted microbial nucleic acid molecules with one or more recombinant CXXC-domain proteins to form a protein-DNA binding reaction; (b) incubating said protein-DNA binding reaction under conditions that promote an interaction between said recombinant CXXC-domain proteins and non-methylated CpG motifs of said one or more nucleosome-depleted microbial nucleic acid molecules; (c) separating unbound recombinant CXXC-domain proteins and recombinant CXXC-domain proteins bound to said non-methylated CpG nucleic acid fragments from a remainder of the protein-DNA binding reaction components; (d) washing said recombinant CXXC-domain proteins bound to said non-methylated CpG nucleic acid fragments, thereby generating one or more enriched nucleic acid molecules for amplification. In some embodiments, washing is configured to remove non-specifically associated nucleic acid molecules and said remainder of said protein-DNA binding reaction components. In some embodiments, amplification comprises performing a polymerase chain reaction (PCR) or derivatives thereof. In some embodiments, the derivatives thereof comprise inverse PCR, anchored PCR, primer-directed rolling circle amplification, or any combination thereof. In some embodiments, the one or more mammalian nucleic acid molecules, and said one or more microbial nucleic acid molecules are derived from one or more liquid biological samples of said one or more subjects. In some embodiments, the one or more subjects comprise human, non-human mammal, or any combination thereof subjects. In some embodiments, the mammalian nucleosome-depleted nucleic acid molecule sequencing reads are obtained from mammalian nucleosome-depleted nucleic acid molecules of the subject's biological sample, where the biological sample comprises one or more mammalian nucleic acid molecules comprising DNA, RNA, cell-free RNA, cell-free DNA, exosomal DNA, exosomal RNA, or any combination thereof, and wherein said one or more microbial nucleic acid molecules comprise microbial cell-free DNA, microbial cell-free RNA, microbial DNA, microbial RNA, or any combination thereof. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the non-oncologic disease comprises healthy, disease, or any combination thereof non-cancer state. In some embodiments, the disease state comprises benign neoplasms of the integumentary, skeletal, muscular, nervous, endocrine, cardiovascular, lymphatic, digestive, respiratory, urinary, reproductive, or any system combination thereof.

In some embodiments, the cancer comprises a cancer of stage I, II, or III.

In some embodiments, the trained predictive model is trained with one or more metagenomic features and corresponding health states of one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, said subject's or subjects' health states comprise said subjects' known non-oncologic disease, cancer, or any combination thereof.

Another aspect of disclosure comprises a method of enriching cell-free microbial nucleic acid molecules of a sample. In some embodiments, the method comprises: (a) contacting a sample of one or more cell-free nucleic acid molecules with a first set of one or more probes, wherein said a first set of one or more probes comprise a binding moiety configured to bind to one or more human nucleic acid molecules complexed to one or more proteins; and (b) enriching one or more cell-free microbial nucleic acid molecules of said sample by removing said one or more probes bound to said one or more human nucleic acid molecules complexed to said one or more proteins from said sample. In some embodiments, the one or more proteins comprise one or more histone proteins, one or more regulatory proteins, or any combination thereof. In some embodiments, the sample comprises plasma, serum, whole blood, urine, cerebral spinal fluid, saliva, sweat, tears, exhaled breath condensate, or any combination, dilution, or processed fraction thereof. In some embodiments, the one or more probes comprise one or more antibodies. In some embodiments, the said removing comprises incubating said one or more antibodies bound to said one or more human nucleic acid molecules complexed to one or more proteins with a solid support, wherein said solid support comprises one or more capture reagents configured to bind to said one or more antibodies.

In some embodiments, the method further comprises (c) contacting said enriched cell-free microbial nucleic acid molecules with a second set of one or more probes, wherein said second set of one or more probes are configured to bind to one or more microbial marker genes. In some embodiments, the one or more microbial marker genes comprise ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the one or more microbial marker genes are sequenced to determine a taxonomic, functional, or any combination thereof abundance of microbes. In some embodiments, the sample comprises a liquid biological sample. In some embodiments, the sample originated from a subject. In some embodiments, the subject is human or non-human mammal. In some embodiments, the one or more proteins comprise histone proteins associated with one or more nucleic acid molecules. In some embodiments, the one or more human nucleic acid molecules comprise DNA, RNA, cell-free RNA, cell-free DNA, exosomal RNA, exosomal DNA, or any combination thereof. In some embodiments, the one or more cell-free microbial nucleic acid molecules comprise cell-free microbial DNA, cell-free microbial RNA, microbial RNA, microbial DNA, or any combination thereof. In some embodiments, removing comprises immunoprecipitating said one or more probes bound to said one or more human nucleic acid molecules.

In some embodiments, the method further comprises (d) preparing a single stranded library from said one or more cell-free microbial nucleic acid molecules of said sample. In some embodiments, the first set of one or more probes are coupled to a solid support. In some embodiments, the solid support comprises a bead, magnetic bead, agarose bead, non-magnetic latex, functionalized Sepharose, pH-sensitive polymers or any combination thereof. In some embodiments, the sample comprises one or more human nucleic acid molecules, one or more microbial nucleic acid molecules, or any combination thereof. In some embodiments, said subject's or subjects' health states comprise said subjects' known non-oncologic disease, cancer, or any combination thereof.

Aspects of the disclosure comprise a method of generating a microbial metagenomic feature set to diagnose a disease, the method comprising: (a) providing a plurality of subjects' health states and said plurality of subjects' biological samples, wherein said biological samples comprise mammalian nucleic acid molecules and microbial nucleic acid molecules; (b) removing said mammalian nucleic acid molecules from said biological samples with an affinity capture reagent; (c) sequencing said microbial nucleic acid molecules to generate microbial sequencing reads; and (d) generating said microbial metagenomic feature set to diagnose said disease by combining a metagenomic feature abundances of said microbial sequencing reads and said plurality of subjects' health states. In some embodiments, the metagenomic feature set comprises microbial taxonomic abundance. In some embodiments, the metagenomic feature set comprises computationally inferred microbial biochemical pathways and said microbial biochemical pathways' associated abundances. In some embodiments, the metagenomic feature set comprises microbial phylogenetic marker genes or marker gene fragments thereof.

In some embodiments, the biological sample comprises a liquid biological sample, where the liquid biological sample comprises: plasma, serum, whole blood, urine, cerebral spinal fluid, saliva, sweat, tears, exhaled breath condensate, or any combination, dilution, or processed fraction thereof.

In some embodiments, the step (b) of the method of generating a microbial metagenomic feature set to diagnose said disease comprises: (a) contacting said liquid biological sample with a solid support comprising immobilized anti-nucleosome antibodies to form antibody-nucleosome interaction complexes; (b) separating said solid support from said liquid biological sample to concentrate said antibody-nucleosome interaction complexes; and (c) purifying the remaining one or more nucleosome-depleted microbial nucleic acid molecules. In some embodiments, the anti-nucleosome antibodies are configured to bind to an epitope comprising DNA and one or more histone proteins. In some embodiments, the solid support comprises a magnetic bead, agarose bead, non-magnetic latex, functionalized Sepharose, pH-sensitive polymers or any combination thereof.

In some embodiments, step (b) of said method of generating a microbial metagenomic feature set to diagnose said disease comprises: (a) contacting said liquid biological sample with one or more anti-nucleosome antibodies to form antibody-nucleosome interaction complexes; (b) contacting said antibody-nucleosome interaction complexes with a solid support; (c) separating said solid support from said liquid biological sample to concentrate said antibody-nucleosome interaction complexes; and (d) purifying the remaining nucleosome-depleted microbial nucleic acid molecules. In some embodiments, the anti-nucleosome antibodies comprise a plurality of epitope tags. In some embodiments, the plurality of epitope tags comprises an N- or C-terminal 6×-histidine tag, green fluorescent protein (GFP), myc, hemagglutinin (HA), Fc fusion, biotin or any combination thereof. In some embodiments, the solid support comprises a magnetic bead, agarose bead, non-magnetic latex, functionalized Sepharose, pH-sensitive polymers or any combination thereof. In some embodiments, the solid support comprises covalently immobilized affinity agents. In some embodiments, the affinity reagents comprise streptavidin, antibodies specific for 6×-histidine tag, green fluorescent protein (GFP), myc, hemagglutinin (HA), biotin, or any combination thereof. In some embodiments, the affinity agents comprise anti-species antibodies.

In some embodiments, step (c) of said method of generating a microbial metagenomic feature set to diagnose said disease comprises: (a) generating single-stranded DNA libraries from said microbial nucleic acid molecules; (b) performing shotgun metagenomic sequencing analysis of said single-stranded DNA libraries to produce sequencing reads; (c) filtering said sequencing reads to produce mammalian DNA-depleted microbial sequencing reads; and (d) decontaminating said mammalian DNA-depleted microbial sequencing reads to remove non-endogenous microbial sequencing reads. In some embodiments, the decontaminating comprises in-silico decontamination. In some embodiments, the filtering comprises computationally mapping said sequencing reads to a human reference genome database.

In some embodiments, step (c) of said method of generating a microbial metagenomic feature set to diagnose said disease comprises: (a) amplifying genomic features of said microbial nucleic acid molecules, thereby generating amplified genomic features; (b) sequencing said amplified genomic features to generate sequencing reads; (c) filtering said sequencing reads to produce mitochondrial DNA-depleted microbial sequencing reads; and (d) decontaminating said mitochondrial DNA-depleted microbial sequencing reads to remove non-endogenous microbial sequencing reads. In some embodiments, the decontaminating comprises in-silico decontamination. In some embodiments, the genomic features comprise microbial phylogenetic marker genes or marker gene fragments thereof.

In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) of said method of generating a microbial metagenomic feature set to diagnose said disease comprises enriching said microbial nucleic acid molecules. In some embodiments, enriching comprises: (a) combining said microbial nucleic acid molecules with hybridization probes, wherein said hybridization probes comprise a nucleic acid sequence complementarity to microbial genomic features; (b) incubating said hybridization probes and said microbial nucleic acid molecules under conditions that promote nucleic acid base pairing between target nucleic acid features and said hybridization probes; (c) separating unbound hybridization probes and hybridized probes bound to said microbial nucleic acid molecules; and (d) washing said hybridized probes bound to said microbial nucleic acid molecules, thereby generating enriched microbial nucleic acid molecules. In some embodiments, the washing is configured to remove non-specifically associated nucleic acid molecules and other reaction components. In some embodiments, enriching comprises: (a) combining said microbial nucleic acid molecules with recombinant CXXC-domain proteins to form a protein-DNA binding reaction; (b) incubating said protein-DNA binding reaction under conditions that promote an interaction between said recombinant CXXC-domain proteins and non-methylated CpG motifs of said microbial nucleic acid molecules; (c) separating unbound recombinant CXXC-domain proteins and recombinant CXXC-domain proteins bound to said non-methylated CpG motifs from a remainder of said protein-DNA binding reaction; and (d) washing said recombinant CXXC-domain proteins bound to said non-methylated CpG nucleic acid fragments, thereby generating enriched nucleic acid molecules for amplification. In some embodiments, the washing is configured to remove non-specifically associated nucleic acid molecules and said remainder of protein-DNA binding reaction components. In some embodiments, the amplification comprises performing a polymerase chain reaction or derivatives thereof. In some embodiments, the derivatives thereof comprise inverse PCR, anchored PCR, primer-directed rolling circle amplification, or any combination thereof. In some embodiments, the mammalian nucleic acid molecules and microbial nucleic acid molecules are derived from liquid biological samples of said plurality of subjects. In some embodiments, the plurality of subjects comprises human, non-human mammal, or any combination thereof subjects.

In some embodiments, the mammalian nucleic acid molecules comprise DNA, RNA, cell-free DNA, cell-free RNA, exosomal DNA, exosomal RNA, or any combination thereof nucleic acid molecules, and wherein said microbial nucleic acid molecules comprise microbial cell-free RNA, microbial cell-free DNA, microbial RNA, microbial DNA, or any combination thereof nucleic acid molecules.

In some embodiments, the method further comprises generating a trained predictive model, wherein said trained predictive model is trained with said microbial metagenomic feature set and said health state of said one or more subjects of said plurality of subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, said subject's or subjects' health states comprise said subjects' known non-oncologic disease, cancer, or any combination thereof.

Aspects of the disclosure comprise a method of diagnosing a disease of a subject, the method comprising: (a) providing a liquid biological sample of said subject, wherein said liquid biological sample comprises mammalian nucleic acid molecules and microbial nucleic acid molecules; (b) removing said mammalian nucleic acid molecules from said liquid biological sample with an affinity capture reagent; (c) sequencing a plurality microbial nucleic acid molecules of said liquid biological sample to generate microbial sequencing reads; (d) generating metagenomic feature abundances of said microbial sequencing reads; and (e) outputting said diagnosis of said disease of said subject at least as a result of providing said microbial metagenomic feature abundances as an input to a trained predictive model. In some embodiments, the disease comprises benign neoplasms of the integumentary, skeletal, muscular, nervous, endocrine, cardiovascular, lymphatic, digestive, respiratory, urinary, reproductive, or any system combinations thereof.

Aspects of the disclosure comprise a system for diagnosing a disease of a subject, the system comprising: (a) a processor; and (b) a non-transitory computer readable storage medium including software configured to cause said processor to: (i) receive subjects' mammalian nucleosome-depleted nucleic acid molecule sequencing reads, wherein said mammalian nucleosome-depleted nucleic acid molecule sequencing reads comprise metagenomic features of microbial nucleic acid molecules; and (ii) output a diagnosis of said disease of said subject at least as a result of providing said metagenomic features as an input to a trained predictive model. In some embodiments, the disease comprises benign neoplasms of the integumentary, skeletal, muscular, nervous, endocrine, cardiovascular, lymphatic, digestive, respiratory, urinary, reproductive, or any system combinations thereof. In some embodiments, said subject's or subjects' health states comprise said subjects' known non-oncologic disease, cancer, or any combination thereof.

Aspects of the disclosure comprise a method of generating a microbial metagenomic feature set to diagnose cancer, the method comprising: (a) providing a plurality of subjects' health states and said plurality of subjects' liquid biological samples, wherein said liquid biological samples comprise mammalian nucleic acid molecules and microbial nucleic acid molecules; (b) removing said mammalian nucleic acid molecules from said liquid biological samples with an affinity capture reagent; (c) sequencing said microbial nucleic acid molecules to generate microbial sequencing reads; and (d) generating said microbial metagenomic feature set to diagnose said cancer by combining a metagenomic feature abundances of said microbial sequencing reads and said plurality of subjects' health states. In some embodiments, the metagenomic feature set comprises microbial taxonomic abundance. In some embodiments, the metagenomic feature set comprises computationally inferred microbial biochemical pathways and said microbial biochemical pathways' associated abundances. In some embodiments, the metagenomic feature set comprises microbial phylogenetic marker genes or marker gene fragments thereof. In some embodiments, the liquid biological sample comprises: plasma, serum, whole blood, urine, cerebral spinal fluid, saliva, sweat, tears, exhaled breath condensate, or any combination, dilution, or processed fraction thereof.

In some embodiments, step (b) of the method of generating a microbial metagenomic feature set to diagnose cancer comprises: (a) contacting said liquid biological sample with a solid support comprising immobilized anti-nucleosome antibodies to form antibody-nucleosome interaction complexes; (b) separating said solid support from said liquid biological sample to concentrate said antibody-nucleosome interaction complexes; and (c) purifying the remaining one or more nucleosome-depleted microbial nucleic acid molecules. In some embodiments, the anti-nucleosome antibodies are configured to bind to an epitope comprising DNA and one or more histone proteins. In some embodiments, the solid supports comprise a magnetic bead, agarose bead, non-magnetic latex, functionalized Sepharose, pH-sensitive polymers or any combination thereof.

In some embodiments, step (b) of the method of generating a microbial metagenomic feature set to diagnose cancer comprises: (a) contacting said liquid biological sample with one or more anti-nucleosome antibodies to form antibody-nucleosome interaction complexes; (b) contacting said antibody-nucleosome interaction complexes with a solid support; (c) separating said solid support from said liquid biological sample to concentrate said antibody-nucleosome interaction complexes; and (d) purifying the remaining nucleosome-depleted microbial nucleic acid molecules. In some embodiments, the anti-nucleosome antibodies comprise a plurality of epitope tags. In some embodiments, the plurality of epitope tags comprises an N- or C-terminal 6×-histidine tag, green fluorescent protein (GFP), myc, hemagglutinin (HA), Fc fusion, biotin or any combination thereof. In some embodiments, the solid support comprises a magnetic bead, agarose bead, non-magnetic latex, functionalized Sepharose, pH-sensitive polymers or any combination thereof. In some embodiments, the solid support comprises covalently immobilized affinity agents. In some embodiments, the affinity reagents comprise streptavidin, antibodies specific for 6×-histidine tag, green fluorescent protein (GFP), myc, hemagglutinin (HA), biotin, or any combination thereof. In some embodiments, the affinity agents comprise anti-species antibodies.

In some embodiments, the step (c) of the method of generating a microbial metagenomic feature set to diagnose cancer comprises: (a) generating single-stranded DNA libraries from said microbial nucleic acid molecules; (b) performing shotgun metagenomic sequencing analysis of said single-stranded DNA libraries to produce sequencing reads; (c) filtering said sequencing reads to produce mammalian DNA-depleted microbial sequencing reads; and (d) decontaminating said mammalian DNA-depleted microbial sequencing reads to remove non-endogenous microbial sequencing reads. In some embodiments, the decontaminating comprises in-silico decontamination. In some embodiments, the filtering comprises computationally mapping said sequencing reads to a human reference genome database.

In some embodiments, step (c) of the method of generating a microbial metagenomic feature set to diagnose cancer comprises: (a) amplifying genomic features of said microbial nucleic acid molecules, thereby generating amplified genomic features; (b) sequencing said amplified genomic features to generate sequencing reads; (c) filtering said sequencing reads to produce mitochondrial DNA-depleted microbial sequencing reads; and (d) decontaminating said mitochondrial DNA-depleted microbial sequencing reads to remove non-endogenous microbial sequencing reads. In some embodiments, decontaminating comprises in-silico decontamination. In some embodiments, the genomic features comprise microbial phylogenetic marker genes or marker gene fragments thereof, in some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) of the method of generating a microbial metagenomic feature set to diagnose cancer comprises enriching said microbial nucleic acid molecules In some embodiments, the enriching comprises: (a) combining purified nucleosome-depleted microbial nucleic acid molecules with hybridization probes, wherein said hybridization probes comprise a nucleic acid sequence complementarity to microbial genomic features; (b) incubating said hybridization probes and said nucleosome-depleted microbial nucleic acid molecules under conditions that promote nucleic acid base pairing between target nucleic acid features and said hybridization probes; (c) separating unbound hybridization probes and hybridized probes bound to said microbial nucleic acid molecules; and (d) washing said hybridized probes bound to said microbial nucleic acid molecules, thereby generating enriched microbial nucleic acid molecules. In some embodiments, the washing is configured to remove non-specifically associated nucleic acid molecules and other reaction components.

In some embodiments, enriching comprises: (a) combining purified nucleosome-depleted microbial nucleic acid molecules with recombinant CXXC-domain proteins to form a protein-DNA binding reaction; (b) incubating said protein-DNA binding reaction under conditions that promote an interaction between said recombinant CXXC-domain proteins and non-methylated CpG motifs of said nucleosome-depleted microbial nucleic acid molecules; (c) separating unbound recombinant CXXC-domain proteins and recombinant CXXC-domain proteins bound to said non-methylated CpG motifs from a remainder of said protein-DNA binding reaction; (d) washing said recombinant CXXC-domain proteins bound to said non-methylated CpG nucleic acid fragments, thereby generating enriched nucleic acid molecules for amplification. In some embodiments, the washing is configured to remove non-specifically associated nucleic acid molecules and said remainder of protein-DNA binding reaction components. In some embodiments, the amplification comprises performing a polymerase chain reaction or derivatives thereof. In some embodiments, the derivatives thereof comprise inverse PCR, anchored PCR, primer-directed rolling circle amplification, or any combination thereof. In some embodiments, the mammalian nucleic acid molecules and microbial nucleic acid molecules are derived from liquid biological samples of said plurality of subjects. In some embodiments, the plurality of subjects comprises human, non-human mammal, or any combination thereof subjects. In some embodiments, the mammalian nucleic acid molecules comprise DNA, RNA, cell-free DNA, cell-free RNA, exosomal DNA, exosomal RNA, or any combination thereof nucleic acid molecules, and wherein said microbial nucleic acid molecules comprise microbial cell-free RNA, microbial cell-free DNA, microbial RNA, microbial DNA, or any combination thereof nucleic acid molecules. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the cancer comprises a cancer of stage I, II, or III. In some embodiments, the method of generating a microbial metagenomic feature set to diagnose cancer comprises generating a trained predictive model, wherein said trained predictive model is trained with said microbial metagenomic feature set and said health state of said one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, said subject's or subjects' health states comprise said subjects' known non-oncologic disease, cancer, or any combination thereof.

Aspects of the disclosure comprise a method of diagnosing a cancer of a subject, the method comprising: (a) providing a liquid biological sample of said subject, wherein said liquid biological sample comprises mammalian nucleic acid molecules and microbial nucleic acid molecules; (b) removing said mammalian nucleic acid molecules from said liquid biological sample with an affinity capture reagent; (c) sequencing a plurality microbial nucleic acid molecules of said liquid biological sample to generate microbial sequencing reads; (d) generating metagenomic feature abundances of said microbial sequencing reads; and (e) outputting said diagnosis of said cancer of said subject at least as a result of providing said microbial metagenomic feature abundances as an input to a trained predictive model. In some embodiments, the microbial metagenomic feature sets comprise microbial taxonomic abundance. In some embodiments, the microbial metagenomic feature sets comprise computationally inferred microbial biochemical pathways and their associated abundance. In some embodiments, the microbial metagenomic feature sets comprise microbial phylogenetic marker genes or marker gene fragments thereof. In some embodiments, the liquid biological sample comprises plasma, serum, whole blood, urine, cerebral spinal fluid, saliva, sweat, tears, exhaled breath condensate, or any combination, dilution, or processed fraction thereof.

In some embodiments, step (b) of the method of diagnosing a cancer of a subject comprises: (a) contacting said liquid biological sample with a solid support comprising immobilized anti-nucleosome antibodies, wherein said anti-nucleosome antibodies are configured to form antibody-nucleosome interaction complexes; (b) separating said solid support from said liquid biological sample to concentrate said antibody-nucleosome interaction complexes; and (c) purifying the remaining nucleosome-depleted microbial nucleic acid molecules. In some embodiments, the anti-nucleosome antibodies recognize an epitope comprising DNA and one or more histone proteins. In some embodiments, the solid support comprises a magnetic bead, an agarose bead, non-magnetic latex, functionalized Sepharose, pH-sensitive polymers or any combinations thereof.

In some embodiments, the step (b) of the method of diagnosing a cancer of a subject comprises: (a) contacting said liquid biological sample with anti-nucleosome antibodies to form antibody-nucleosome interaction complexes; (b) contacting said antibody-nucleosome interaction complexes with a solid support configured to bind to said antibody-nucleosome interaction complexes; (c) separating said solid support from said liquid biological sample to concentrate said antibody-nucleosome interaction complexes; and (d) purifying the remaining nucleosome-depleted microbial nucleic acids. In some embodiments, the anti-nucleosome antibodies comprise epitope tags. In some embodiments, the epitope tags comprise an N- or C-terminal 6×-histidine tag, green fluorescent protein (GFP), myc, hemagglutinin (HA), Fc fusion, biotin or any combination thereof. In some embodiments, the solid support comprises a magnetic bead, agarose bead, non-magnetic latex, functionalized Sepharose, pH-sensitive polymers or any combination thereof. In some embodiments, the solid support comprises covalently immobilized affinity agents. In some embodiments, the covalently immobilized affinity agents comprise streptavidin, antibodies specific for 6×-histidine tag, green fluorescent protein (GFP), myc, hemagglutinin (HA), biotin, or any combination thereof. In some embodiments, the covalently immobilized affinity agents comprise anti-species antibodies.

In some embodiments, step (c) of the method of diagnosing a cancer of a subject comprises: (a) generating single-stranded DNA libraries from said microbial nucleic acid molecules; (b) performing shotgun metagenomic sequencing analysis of said single-stranded DNA libraries to produce sequencing reads; (c) filtering said sequencing reads to produce mammalian DNA-depleted microbial sequencing reads; and (d) decontaminating said mammalian DNA-depleted microbial sequencing reads to remove non-endogenous microbial sequencing reads. In some embodiments, the decontaminating comprises in-silico decontamination of said mammalian DNA-depleted microbial sequencing reads. In some embodiments, the filtering comprises computationally mapping said sequencing reads to a human reference genome database.

In some embodiments, the step (c) of the method of diagnosing a cancer of a subject comprises: (a) amplifying genomic features of said microbial nucleic acid molecules, thereby generating amplified genomic features; (b) sequencing said amplified genomic features to generate sequencing reads; (c) filtering said sequencing reads to produce mitochondrial DNA-depleted microbial sequencing reads; and (d) decontaminating said mitochondrial DNA-depleted microbial sequencing reads to remove non-endogenous microbial sequencing reads. In some embodiments, the decontaminating comprises in-silico decontamination of said mitochondrial DNA-depleted microbial sequencing reads. In some embodiments, the genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, the amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) of the method of diagnosing a cancer of a subject comprises enriching said microbial nucleic acid molecules. In some embodiments, the enriching of said microbial nucleic acid molecules comprises: (a) combining purified nucleosome-depleted microbial nucleic acid molecules with hybridization probes, wherein said hybridization probes comprise a nucleic acid sequence complementarity to microbial genomic nucleic acid features; (b) incubating said hybridization probes and said nucleosome-depleted microbial nucleic acid molecules under conditions that promote nucleic acid base pairing between said microbial genomic nucleic acid features and said hybridization probes; (c) separating unbound hybridization probes and hybridized probes bound to said nucleosome-depleted microbial nucleic acid molecules; and (d) washing said hybridized probes bound to said nucleosome-depleted microbial nucleic acid molecules, thereby generating one or more enriched microbial nucleic acid molecules. In some embodiments, the washing is configured to remove non-specifically associated nucleic acid molecules and other reaction components

In some embodiments, the enriching said microbial nucleic acid molecules comprises: (a) combining purified nucleosome-depleted microbial nucleic acid molecules with recombinant CXXC-domain proteins to form a protein-DNA binding reaction; (b) incubating said protein-DNA binding reaction under conditions that promote an interaction between said recombinant CXXC-domain proteins and non-methylated CpG motifs of said nucleosome-depleted microbial nucleic acid molecules; (c) separating unbound recombinant CXXC-domain proteins and recombinant CXXC-domain proteins bound to said non-methylated CpG nucleic acid fragments from a remainder of the protein-DNA binding reaction components; (d) washing said recombinant CXXC-domain proteins bound to said non-methylated CpG nucleic acid fragments, thereby generating enriched nucleic acid molecules for amplification. In some embodiments, the washing is configured to remove non-specifically associated nucleic acid molecules and said remainder of said protein-DNA binding reaction components. In some embodiments, the amplification comprises performing a polymerase chain reaction or derivatives thereof. In some embodiments, the derivatives thereof comprise inverse PCR, anchored PCR, primer-directed rolling circle amplification, or any combination thereof. In some embodiments, the mammalian nucleic acid molecules and said microbial nucleic acid molecules are derived from liquid biological samples of said subject. In some embodiments, the subjects comprise a human, non-human mammal, or any combination thereof subject. In some embodiments, the mammalian nucleic acid molecules comprise DNA, RNA, cell-free RNA, cell-free DNA, exosomal DNA, exosomal RNA, or any combination thereof nucleic acid molecules, and wherein said microbial nucleic acid molecules comprise microbial cell-free DNA, microbial cell-free RNA, microbial DNA, microbial RNA, or any combination thereof nucleic acid molecules. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the cancer comprises a cancer of stage I, II, or III. In some embodiments, the trained predictive model is trained with microbial metagenomic feature sets and corresponding health state of one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, the subject is suspected of having cancer or a disease. In some embodiments, the subject's imaging results indicate a potential presence of cancer. In some embodiments, said subject's or subjects' health states comprise said subjects' known non-oncologic disease, cancer, or any combination thereof.

Aspects of the disclosure comprise a system for diagnosing cancer of a subject, the system comprising: (a) a processor; and (b) a non-transitory computer readable storage medium including software configured to cause said processor to: (i) receive subjects' mammalian nucleosome-depleted nucleic acid molecule sequencing reads, wherein said mammalian nucleosome-depleted nucleic acid molecule sequencing reads comprise metagenomic features of microbial nucleic acid molecules; and (ii) output a diagnosis of said cancer of said subject at least as a result of providing said metagenomic features as an input to a trained predictive model. In some embodiments, the metagenomic features comprise microbial taxonomic abundance. In some embodiments, the metagenomic features comprise computationally inferred microbial biochemical pathways and their associated abundance. In some embodiments, the metagenomic features comprise microbial phylogenetic marker genes or marker gene fragments thereof. In some embodiments, the liquid biological sample comprises: plasma, serum, whole blood, urine, cerebral spinal fluid, saliva, sweat, tears, exhaled breath condensate, or any combination, dilution, or processed fraction thereof. In some embodiments, the mammalian nucleosome-depleted nucleic acid molecules' sequencing reads are produced by: (a) contacting said liquid biological sample with a solid support to form antibody-nucleosome interaction complexes, wherein said solid support comprises a surface comprising anti-nucleosome antibodies coupled thereto; (b) separating said solid support from said liquid biological sample to concentrate said antibody-nucleosome interaction complexes; (c) purifying the remaining nucleosome-depleted microbial nucleic acid molecules; and (d) sequencing said purified nucleosome-depleted microbial nucleic acid molecules. In some embodiments, the anti-nucleosome antibodies are configured to recognize an epitope comprising DNA and histone proteins. In some embodiments, the solid support comprises a magnetic bead, an agarose bead, non-magnetic latex, functionalized Sepharose, pH-sensitive polymers or any combinations thereof.

In some embodiments, the mammalian nucleosome-depleted nucleic acid molecules' sequencing reads are produced by: (a) contacting said liquid biological sample with anti-nucleosome antibodies to form antibody-nucleosome interaction complexes; (b) contacting said antibody-nucleosome interaction complexes with a solid support; (c) separating said solid support from said liquid biological sample to concentrate said antibody-nucleosome interaction complexes; (d) purifying the remaining nucleosome-depleted microbial nucleic acid molecules; and (e) sequencing said purified one or more nucleosome-depleted microbial nucleic acid molecules. In some embodiments, the anti-nucleosome antibodies comprise epitope tags. In some embodiments, the epitope tags comprise an N- or C-terminal 6×-histidine tag, green fluorescent protein (GFP), myc, hemagglutinin (HA), Fc fusion, biotin or any combination thereof. In some embodiments, the solid support comprises a magnetic bead, agarose bead, non-magnetic latex, functionalized Sepharose, pH-sensitive polymers or any combination thereof. In some embodiments, the solid support comprises covalently immobilized affinity agents. In some embodiments, the covalently immobilized affinity agents comprise streptavidin, antibodies specific for 6×-histidine tag, green fluorescent protein (GFP), myc, hemagglutinin (HA), biotin, or any combination thereof. In some embodiments, the covalently immobilized affinity agents comprise anti-species antibodies.

In some embodiments, the mammalian nucleosome-depleted nucleic acid molecules' sequencing reads are produced by: (a) generating single-stranded DNA libraries from said microbial nucleic acid molecules; (b) performing shotgun metagenomic sequencing analysis of said single-stranded DNA libraries to produce sequencing reads; (c) filtering said sequencing reads to produce mammalian DNA-depleted microbial sequencing reads; and (d) decontaminating said mammalian DNA-depleted microbial sequencing reads to remove non-endogenous microbial sequencing reads. In some embodiments, decontaminating comprises in-silico decontamination of said mammalian DNA-depleted microbial sequencing reads. In some embodiments, filtering comprises computationally mapping said sequencing reads to a human reference genome database.

In some embodiments, the mammalian nucleosome-depleted nucleic acid molecules' sequencing reads are produced by: (a) amplifying genomic features of said one or more microbial nucleic acid molecules, thereby generating amplified genomic features; (b) sequencing said amplified genomic features to generate sequencing reads; (c) filtering said sequencing reads to produce mitochondrial DNA-depleted microbial sequencing reads; and (d) decontaminating said mitochondrial DNA-depleted microbial sequencing reads to remove non-endogenous microbial sequencing reads. In some embodiments, decontaminating comprises in-silico decontamination of said mitochondrial DNA-depleted microbial sequencing reads. In some embodiments, the genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction (PCR) or derivatives thereof. In some embodiments, the derivatives thereof comprise inverse PCR, anchored PCR, primer-directed rolling circle amplification, or any combination thereof. In some embodiments, the microbial nucleic acid molecules are enriched from said mammalian nucleosome-depleted nucleic acid molecules.

In some embodiments, the enriching of said microbial nucleic acid molecules comprises: (a) combining purified nucleosome-depleted microbial nucleic acid molecules with hybridization probes, wherein said hybridization probes comprise a nucleic acid sequence complementarity to microbial genomic nucleic acid features; (b) incubating said hybridization probes and nucleosome-depleted microbial nucleic acid molecules under conditions that promote nucleic acid base pairing between said microbial genomic nucleic acid features and said hybridization probes; (c) separating unbound hybridization probes and hybridized probes bound to said nucleosome-depleted microbial nucleic acid molecules; and (d) washing said hybridized probes bound to said nucleosome-depleted microbial nucleic acid molecules, thereby generating enriched microbial nucleic acid molecules. In some embodiments, washing is configured to remove non-specifically associated nucleic acid molecules and other reaction components

In some embodiments, enriching of said microbial nucleic acid molecules comprises: (a) combining purified nucleosome-depleted microbial nucleic acid molecules with recombinant CXXC-domain proteins to form a protein-DNA binding reaction; (b) incubating said protein-DNA binding reaction under conditions that promote an interaction between said recombinant CXXC-domain proteins and non-methylated CpG motifs of said nucleosome-depleted microbial nucleic acid molecules; (c) separating unbound recombinant CXXC-domain proteins and recombinant CXXC-domain proteins bound to said non-methylated CpG nucleic acid fragments from a remainder of the protein-DNA binding reaction components; and (d) washing said recombinant CXXC-domain proteins bound to said non-methylated CpG nucleic acid fragments, thereby generating enriched nucleic acid molecules for amplification. In some embodiments, washing is configured to remove non-specifically associated nucleic acid molecules and said remainder of said protein-DNA binding reaction components. In some embodiments, amplification comprises performing a polymerase chain reaction (PCR) or derivatives thereof. In some embodiments, the derivatives thereof comprise inverse PCR, anchored PCR, primer-directed rolling circle amplification, or any combination thereof. In some embodiments, the mammalian nucleic acid molecules, and said microbial nucleic acid molecules are derived from liquid biological samples of said subject. In some embodiments, the subject comprises human, non-human mammal, or any combination thereof subjects. In some embodiments, the mammalian nucleic acid molecules comprise DNA, RNA, cell-free RNA, cell-free DNA, exosomal DNA, exosomal RNA, or any combination thereof, and wherein said microbial nucleic acid molecules comprise microbial cell-free DNA, microbial cell-free RNA, microbial DNA, microbial RNA, or any combination thereof. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof. In some embodiments, the cancer comprises a cancer of stage I, II, or III. In some embodiments, the trained predictive model is trained with metagenomic features and corresponding health states of a plurality of subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, the subject is suspected of having cancer or a disease. In some embodiments, the subject's imaging results indicate a potential presence of cancer.

Aspects of the disclosure comprise a method of generating metagenomic features of a sample of cell-free microbial nucleic acid molecules to diagnose a non-oncologic disease, comprising: (a) contacting said sample of cell-free nucleic acid molecules with a probe, wherein said probe comprises a binding moiety configured to bind to human nucleic acid molecules complexed to proteins; (b) removing said probe bound to said human nucleic acid molecules complexed to said proteins thereby producing enriched cell-free microbial nucleic acid molecules; and (c) generating metagenomic features of said enriched cell-free microbial nucleic acid molecules configured to diagnose a non-oncologic disease. In some embodiments, the proteins comprise one or more histone proteins, one or more regulatory proteins, or any combination thereof. In some embodiments, the sample comprises plasma, serum, whole blood, urine, cerebral spinal fluid, saliva, sweat, tears, exhaled breath condensate, or any combination, dilution, or processed fraction thereof. In some cases, the probes comprise one or more antibodies. In some embodiments, removing comprises incubating said antibodies bound to said human nucleic acid molecules complexed to said proteins with a solid support, wherein said solid support comprises capture reagents configured to bind to said antibodies.

In some embodiments, step (c) of the method of generating metagenomic features of a sample of cell-free microbial nucleic acid molecules to diagnose a non-oncologic disease comprises contacting said enriched cell-free microbial nucleic acid molecules with a second set of probes, wherein said second set of probes are configured to bind to microbial marker genes. In some embodiments, the microbial marker genes comprise ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the microbial marker genes are sequenced to determine a taxonomic, functional, or any combination thereof abundance of microbes. In some embodiments, the sample comprises a liquid biological sample. In some embodiments, the sample originated from a subject. In some embodiments, the subject is human or non-human mammal. In some embodiments, the proteins comprise histone proteins associated with nucleic acid molecules. In some embodiments, the human nucleic acid molecules comprise DNA, RNA, cell-free RNA, cell-free DNA, exosomal RNA, exosomal DNA, or any combination thereof. In some embodiments, the cell-free microbial nucleic acid molecules comprise cell-free microbial DNA, cell-free microbial RNA, microbial RNA, microbial DNA, or any combination thereof. In some embodiments, removing comprises immunoprecipitating said probes bound to said human nucleic acid molecules. In some embodiments, the method of generating metagenomic features of a sample of cell-free microbial nucleic acid molecules to diagnose a non-oncologic disease further comprising (d) preparing a single stranded library from said cell-free microbial nucleic acid molecules of said sample. In some embodiments, the first set of probes are coupled to a solid support. In some embodiments, the solid support comprises a bead, magnetic bead, agarose bead, non-magnetic latex, functionalized Sepharose, pH-sensitive polymers or any combination thereof. In some embodiments, the sample comprises human nucleic acid molecules, microbial nucleic acid molecules, or any combination thereof. In some embodiments, the non-oncologic disease comprises benign neoplasms of the integumentary, skeletal, muscular, nervous, endocrine, cardiovascular, lymphatic, digestive, respiratory, urinary, reproductive, or any system combinations thereof. In some embodiments, said subject's or subjects' health states comprise said subjects' known non-oncologic disease, cancer, or any combination thereof.

Aspects of the disclosure comprise a method of generating a microbial metagenomic feature set to diagnose a non-oncologic disease, the method comprising: (a) providing a plurality of subjects' health states and said plurality of subjects' liquid biological samples, wherein said liquid biological samples comprise mammalian nucleic acid molecules and microbial nucleic acid molecules; (b) removing said mammalian nucleic acid molecules from said liquid biological samples with an affinity capture reagent; (c) sequencing said microbial nucleic acid molecules to generate microbial sequencing reads; and (d) generating said microbial metagenomic feature set to diagnose a non-oncologic disease by combining a metagenomic feature abundances of said microbial sequencing reads and said plurality of subjects' health states. In some embodiments, the metagenomic feature set comprises microbial taxonomic abundance. In some embodiments, the metagenomic feature set comprises computationally inferred microbial biochemical pathways and said microbial biochemical pathways' associated abundances. In some embodiments, the metagenomic feature set comprises microbial phylogenetic marker genes or marker gene fragments thereof. In some embodiments, the liquid biological sample comprises: plasma, serum, whole blood, urine, cerebral spinal fluid, saliva, sweat, tears, exhaled breath condensate, or any combination, dilution, or processed fraction thereof.

In some embodiments, step (b) of the method of generating a microbial metagenomic feature set to diagnose a non-oncologic disease comprises: (a) contacting said liquid biological sample with a solid support comprising immobilized anti-nucleosome antibodies to form antibody-nucleosome interaction complexes; (b) separating said solid support from said liquid biological sample to concentrate said antibody-nucleosome interaction complexes; and (c) purifying the remaining one or more nucleosome-depleted microbial nucleic acid molecules. In some embodiments, the anti-nucleosome antibodies are configured to bind to an epitope comprising DNA and one or more histone proteins. In some embodiments, the solid support comprises a magnetic bead, agarose bead, non-magnetic latex, functionalized Sepharose, pH-sensitive polymers or any combination thereof.

In some embodiments, the step (b) of the method of generating a microbial metagenomic feature set to diagnose a non-oncologic disease comprises: (a) contacting said liquid biological sample with one or more anti-nucleosome antibodies to form antibody-nucleosome interaction complexes; (b) contacting said antibody-nucleosome interaction complexes with a solid support; (c) separating said solid support from said liquid biological sample to concentrate said antibody-nucleosome interaction complexes; and (d) purifying the remaining nucleosome-depleted microbial nucleic acid molecules. In some embodiments, the anti-nucleosome antibodies comprise a plurality of epitope tags. In some embodiments, the plurality of epitope tags comprises an N- or C-terminal 6×-histidine tag, green fluorescent protein (GFP), myc, hemagglutinin (HA), Fc fusion, biotin or any combination thereof. In some embodiments, the solid support comprises a magnetic bead, agarose bead, non-magnetic latex, functionalized Sepharose, pH-sensitive polymers or any combination thereof. In some embodiments, the solid support comprises covalently immobilized affinity agents. In some embodiments, the affinity reagents comprise streptavidin, antibodies specific for 6×-histidine tag, green fluorescent protein (GFP), myc, hemagglutinin (HA), biotin, or any combination thereof. In some embodiments, the affinity agents comprise anti-species antibodies.

In some embodiments, step (c) of the method of generating a microbial metagenomic feature set to diagnose a non-oncologic disease comprises: (a) generating single-stranded DNA libraries from said microbial nucleic acid molecules; (b) performing shotgun metagenomic sequencing analysis of said single-stranded DNA libraries to produce sequencing reads; (c) filtering said sequencing reads to produce mammalian DNA-depleted microbial sequencing reads; and (d) decontaminating said mammalian DNA-depleted microbial sequencing reads to remove non-endogenous microbial sequencing reads. In some embodiments, the decontaminating comprises in-silico decontamination. In some embodiments, the filtering comprises computationally mapping said sequencing reads to a human reference genome database.

In some embodiments, step (c) of the method of generating a microbial metagenomic feature set to diagnose a non-oncologic disease comprises: (a) amplifying genomic features of said microbial nucleic acid molecules, thereby generating amplified genomic features; (b) sequencing said amplified genomic features to generate sequencing reads;
filtering said sequencing reads to produce mitochondrial DNA-depleted microbial sequencing reads; and (c) decontaminating said mitochondrial DNA-depleted microbial sequencing reads to remove non-endogenous microbial sequencing reads. In some embodiments, decontaminating comprises in-silico decontamination. In some embodiments, the genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some embodiments, the fungal marker genes comprise one or more of: ribosomal RNA gene 18S, ribosomal RNA gene 5.8S, ribosomal RNA gene 28S, and the internal transcribed spacer regions 1 and 2. In some embodiments, the microbial phylogenetic marker genes comprise bacterial, fungal, or any combination thereof marker genes. In some embodiments, amplifying comprises performing a polymerase chain reaction or derivatives thereof. In some embodiments, the derivatives thereof comprise inverse PCR, anchored PCR, primer-directed rolling circle amplification, or any combination thereof.

In some embodiments, step (c) of the method of generating a microbial metagenomic feature set to diagnose a non-oncologic disease comprises enriching said microbial nucleic acid molecules. In some embodiments, enriching comprises: (a) combining purified nucleosome-depleted microbial nucleic acid molecules with hybridization probes, wherein said hybridization probes comprise a nucleic acid sequence complementarity to microbial genomic features; (b) incubating said hybridization probes and said nucleosome-depleted microbial nucleic acid molecules under conditions that promote nucleic acid base pairing between target nucleic acid features and said hybridization probes; (c) separating unbound hybridization probes and hybridized probes bound to said microbial nucleic acid molecules; and (d) washing said hybridized probes bound to said microbial nucleic acid molecules, thereby generating enriched microbial nucleic acid molecules. In some embodiments, washing is configured to remove non-specifically associated nucleic acid molecules and other reaction components.

In some embodiments, enriching comprises: (a) combining purified nucleosome-depleted microbial nucleic acid molecules with recombinant CXXC-domain proteins to form a protein-DNA binding reaction; (b) incubating said protein-DNA binding reaction under conditions that promote an interaction between said recombinant CXXC-domain proteins and non-methylated CpG motifs of said nucleosome-depleted microbial nucleic acid molecules; and (c) separating unbound recombinant CXXC-domain proteins and recombinant CXXC-domain proteins bound to said non-methylated CpG motifs from a remainder of said protein-DNA binding reaction; and (d) washing said recombinant CXXC-domain proteins bound to said non-methylated CpG nucleic acid fragments, thereby generating enriched nucleic acid molecules for amplification. In some embodiments, washing is configured to remove non-specifically associated nucleic acid molecules and said remainder of protein-DNA binding reaction components. In some embodiments, amplification comprises performing a polymerase chain reaction or derivatives thereof. In some embodiments, the derivatives thereof comprise inverse PCR, anchored PCR, primer-directed rolling circle amplification, or any combination thereof. In some embodiments, the mammalian nucleic acid molecules and microbial nucleic acid molecules are derived from liquid biological samples of said plurality of subjects. In some embodiments, the plurality of subjects comprises human, non-human mammal, or any combination thereof subjects. In some embodiments, the mammalian nucleic acid molecules comprise DNA, RNA, cell-free DNA, cell-free RNA, exosomal DNA, exosomal RNA, or any combination thereof nucleic acid molecules, and wherein said microbial nucleic acid molecules comprise microbial cell-free RNA, microbial cell-free DNA, microbial RNA, microbial DNA, or any combination thereof nucleic acid molecules.

In some embodiments, the method of generating a microbial metagenomic feature set to diagnose a non-oncologic disease comprises generating a trained predictive model, wherein said trained predictive model is trained with said microbial metagenomic feature set and said health state of said one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, the machine learning model comprises a machine learning classifier. In some embodiments, the machine learning model comprises a gradient boosting machine, neural network, support vector machine, k-means, classification trees, random forest, regression, or any combination thereof machine learning models. In some embodiments, the non-oncologic disease comprises benign neoplasms of the integumentary, skeletal, muscular, nervous, endocrine, cardiovascular, lymphatic, digestive, respiratory, urinary, reproductive, or any system combinations thereof.

In some embodiments, said subject's or subjects' health states comprise said subjects' known non-oncologic disease, cancer, or any combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow diagram of enriching cell-free microbial nucleic acid molecules, as described in some embodiments herein.

FIGS. 2A-2B show flow diagrams of enriching cell-free microbial nucleic acid molecules with a plurality of nucleosome antibodies coupled to a solid support (FIG. 2A) and a plurality of nucleosome-specific antibodies not coupled to a solid support (FIG. 2B), as described in some embodiments herein.

FIGS. 3A-3B show flow diagrams of generating microbial taxonomic (FIG. 3A) and microbial functional (FIG. 3B) machine learning feature sets using enriched cell free microbial nucleic acid molecules, as described in some embodiments herein.

FIGS. 4A-4B show flow diagrams of generating one or more microbial taxonomy (FIG. 4A) and/or microbial functional pathway (FIG. 4B) machine learning (ML) diagnostic model classifiers from nucleosome depleted samples from healthy, cancerous, and non-cancerous healthy subjects, as described in some embodiments herein.

FIG. 5 shows a flow diagram of hybridization and/or protein-based enrichment of cell free microbial nucleic acid molecules' biomarker genes, as described in some embodiments herein.

FIGS. 6A-6C show flow diagrams of generating microbial taxonomy (FIG. 6A), microbial functional (FIG. 6B), and/or microbial amplicon sequence variants (ASV) (FIG. 6C) machine learning feature sets using targeted microbial amplicon sequencing of cell free microbial nucleic acid molecules.

FIG. 7 shows a computer system configured to implement the methods of the disclosure, as described in some embodiments herein.

### DETAILED DESCRIPTION

It has been shown that diseases, e.g., cancer, have unique microbial (e.g. bacterial, viral, and/or fungal) abundances that can serve as a diagnostic or screening proxy for cancer, cancer stage, cancer organ of origin, cancer sub-type, or any combination thereof. Such cancer associated and/or correlated microbial abundance(s) may be determined through minimally invasive screening and/or diagnostics, e.g., detecting non-human nucleic acid molecule abundance of cell-free microbial nucleic acids in a subject's biological sample (e.g., solid and/or liquid biological samples). Detecting microbial abundances from cell-free microbial nucleic acid molecules of a subject's biological sample faces many problems due to the inherently small quantity of cell-free microbial nucleic acid molecules present amongst a large quantity of host human nucleic acid molecules. Thus, there is a need of methods and/or systems configured to deplete host human nucleic acid molecules (e.g., human DNA and/or RNA) of a biological sample to enrich the comparatively small quantity of cell-free microbial nucleic acid molecules present in biological samples. The abundance of the enriched cell free microbial nucleic acid molecules may be used to generate one or more features, as described elsewhere herein, correlated and/or associated with a health state (e.g., cancerous, non-cancerous disease, or healthy) of a subject from which the cell free microbial nucleic acid molecules were obtained and/or collected from. In some cases, a predictive model (e.g., an artificial intelligence model, machine learning model, etc.) may be trained with the one or more features of microbial nucleic acid molecule abundance, where the trained predictive model may be used to predict, monitor, and/or diagnose a health state of a subject's biological sample microbial nucleic acid molecule abundance(s) not used to train the predictive model.

Provided herein are methods and systems that may be configured to enrich microbial nucleic acid molecules and/or deplete human nucleic acid molecules of a biological sample of a subject used for disease screening and/or diagnostic analysis, as described elsewhere herein. The enrichment of microbial nucleic acid molecules and/or the depletion of human nucleic acid molecules may increase in accuracy, specificity, and/or sensitivity, of a diagnostic, screening, and/or predictive result provided by a predictive model, described elsewhere herein, by at least about 1%, at least about 2%, at least about 3%, at least about 4%, at least about 5%, at least about 6%, at least about 7%, at least about 8%, at least about 9%, at least about 10%, at least about 15%, or at least about 20% compared to disease screening and/or diagnostic analysis that do not utilize the cell-free microbial nucleic acid molecule enrichment and/or human nucleic acid molecule depletion, as described elsewhere herein.

In some cases, the disclosure provided herein describes methods and/or systems configured to determine, identify, classify, and/or generate one or more microbial nucleic acid molecule features of one or more subjects' enriched microbial nucleic acid molecules derived from the subjects' biological sample that may differentiate, classify, screen for, and/or diagnose a health state of the one or more subjects and/or one or more groups of subjects. In some cases the biological samples may comprise a liquid biological sample, tissue biological sample, or a combination thereof. In some cases, the one or more nucleic acid molecule features of the one or more subjects may be used to train a predictive model, as described elsewhere herein. In some cases, the one or more nucleic acid molecule features may be derived, obtained, received, and/or determined from one or more enriched microbial nucleic acid molecules of one or more biological samples of a subject and/or a plurality of subjects. In some instances, the microbial nucleic acid molecules may comprise one or more nucleic acid molecules from bacteria, fungi, viruses, or any combination thereof. In some cases, the health state of the one or more subjects, as described elsewhere herein, may comprise a cancerous health state, a non-cancerous disease health state, or healthy health state (i.e., where the subject does not have cancer or a non-cancerous disease). In some instances, the cancerous health state may comprise an individual with cancer. In some cases, the cancer may comprise lung, breast, ovarian, gastro-intestinal, head and neck, liver, pancreas, prostate, skin, or any combination thereof cancers. In some cases, the lung cancer may comprise non-small cell lung cancer. In some cases, the cancerous health state may comprise a diagnosis of a cancer's stage (e.g., Stage I, Stage II, Stage II, etc.). In some cases the health state may comprise a spatial location (i.e., an anatomical location) of the cancer and/or disease within the subject or plurality of subjects. In some cases, the health state may comprise a tissue and/or organ of origin of the cancer. In some cases, the non-cancerous disease health state may comprise lung disease. In some instances, lung disease may comprise: carcinoid, hamartoma, granuloma, interstitial fibrosis, emphysema, bronchitis, chronic obstructive pulmonary disease, pneumonia, sarcoidosis, or any combination thereof. In some cases, the liquid biological sample may comprise a liquid biopsy. In some cases, the liquid biopsy may comprise plasma, serum, whole blood, urine, cerebral spinal fluid, saliva, sweat, tears, exhaled breath condensate, or any combination thereof. In some cases, the tissue biological sample may comprise a tissue biopsy of one or more regions, organs, and/or anatomical locations of a subject (e.g., lung, skin, liver, pancreas, brain, etc.).

### Methods

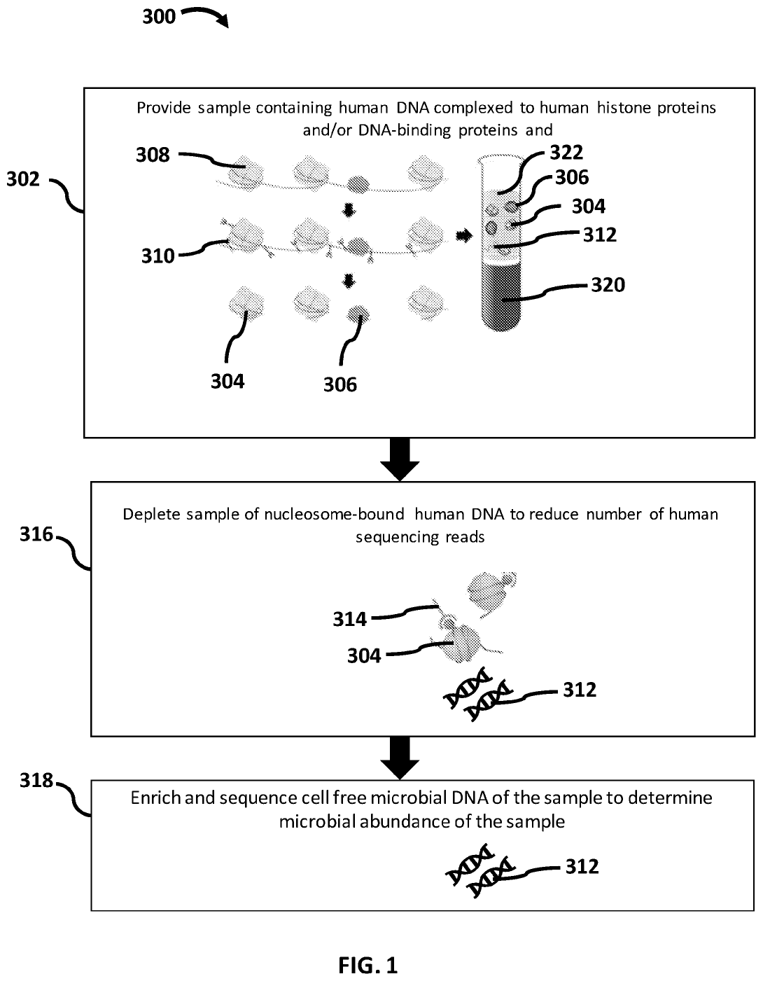

In some cases, the disclosure provides a method of enriching microbial nucleic acid molecules 300, as shown in FIG. 1. In some cases, the method may comprise: receiving, providing and/or obtaining a biological sample containing one or more nucleic acid molecules (308, 306, 304, 312) (e.g., microbial 312 and/or human nucleic acid molecules) 302; depleting the biological sample of protein (e.g., histone proteins) 308 coupled to one or more human nucleic acid molecules 304 by removing affinity-based probes 314 coupled to the protein-human DNA complex (316), thereby enriching the microbial nucleic acid molecules 312 of the biological sample 318. In some cases, one or more endonucleases 310 may be used to cleave or separate one or more segments of human nucleic acid molecules (e.g., DNA) bound to the protein(s). In some instances, the proteins may comprise transcription factors 306 that may comprise human DNA coupled thereto. In some cases, the biological sample may comprise a liquid biological sample, tissue biological sample, or any combination thereof. In some cases, the liquid biological sample may comprise whole blood 320, plasma 322, serum, urine, cerebral spinal fluid, saliva, sweat, tears, exhaled breath condensate, or any combination, dilution, and/or processed fraction thereof. In some cases, plasma 322 may be obtained, isolated, and/or separated from whole blood 320 by centrifugation. In some cases, the microbial nucleic acid molecules 312 may comprise nucleic acid molecules originating from bacterial, viral, fungal, or any combination thereof origins. In some instances, the microbial nucleic acid molecules may comprise cell-free microbial nucleic acid molecules (e.g., cell-free microbial DNA and/or RNA).

In some cases, the affinity-based probes may comprise antibodies, where the antibodies may comprise a binding motif configured to couple to one or more regions and/or surfaces of the protein-human nucleic acid molecule (e.g., DNA) complex. In some cases, the binding motif of the antibodies may couple to an epitope comprising human DNA and one or more histone proteins. In some cases, the antibodies may comprise anti-nucleosome antibodies. In some instances, the antibodies may be bound to a solid support. The solid support may comprise a magnetic bead, agarose bead, non-magnetic latex, functionalize Sepharose, pH-sensitive polymers, or any combination thereof.

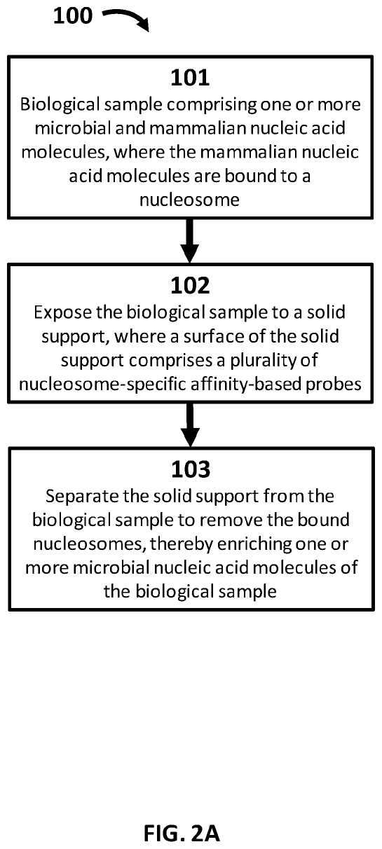

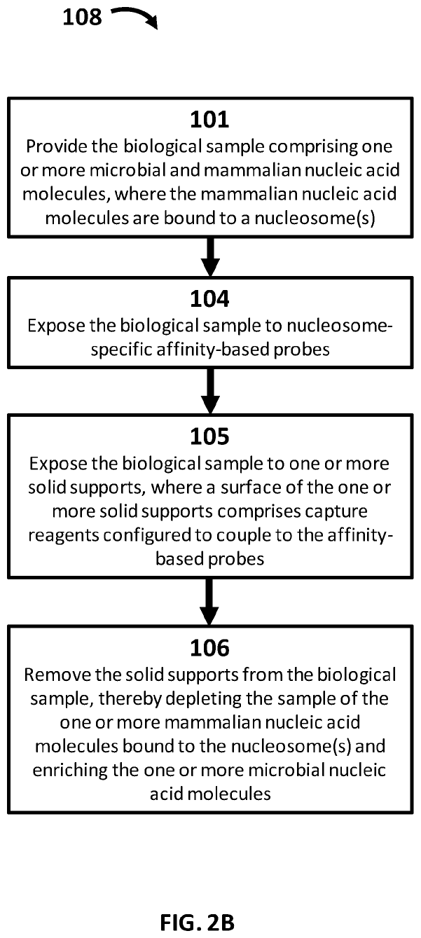

In some embodiments, provided herein is a method of depleting a biological sample of human nucleic acid molecules bound to nucleosomes with an affinity-based probe coupled to a surface of a solid support, as described elsewhere herein, coupled to a solid support 100, as shown in FIG. 2A. In some instances, the method may comprise the steps of: providing, obtaining, and/or collecting a biological sample (e.g., a liquid biological sample), where the biological sample comprises one or more microbial and mammalian nucleic acid molecules 101; expose the biological sample to a solid support, where a surface of the solid support comprises one or more nucleosome-specific affinity-based probes 102; and separating the solid support from the biological sample to remove the bound nucleosomes coupled to the plurality of nucleosome-specific affinity-based probes, thereby enriching one or more microbial nucleic acid molecules of the biological sample 103. In some cases, the biological sample may comprise whole blood. In some instances, the solid support may be exposed to plasma of a whole blood sample.

In some embodiments, provided herein is a method of depleting a biological sample of human nucleic acid molecules bound to nucleosomes with an affinity-based probe configured to couple to a solid support 108, as shown in FIG. 2B. In some cases, the method may comprise the steps of: providing, obtaining, and/or collecting a biological sample comprising one or more microbial and mammalian nucleic acid molecules, where the mammalian nucleic acid molecules are bound to nucleosome(s) 101; exposing the biological sample to epitope and/or affinity-tagged nucleosome-specific affinity-based probes, described elsewhere herein 104; exposing the biological sample to one or more solid supports, where a surface of the one or more solid supports comprises capture molecules configured to couple to the one or more affinity-based probes 105; removing the solid supports from the biological sample, thereby depleting the sample of the one or more mammalian nucleic acid molecules bound to the nucleosome(s) and thereby enriching the one or more microbial nucleic acid molecules 106. In some cases, the capture molecules may comprise anti-species antibodies configured to couple and/or bind to human and/or non-human mammal (e.g., rabbit, mouse) antibodies.

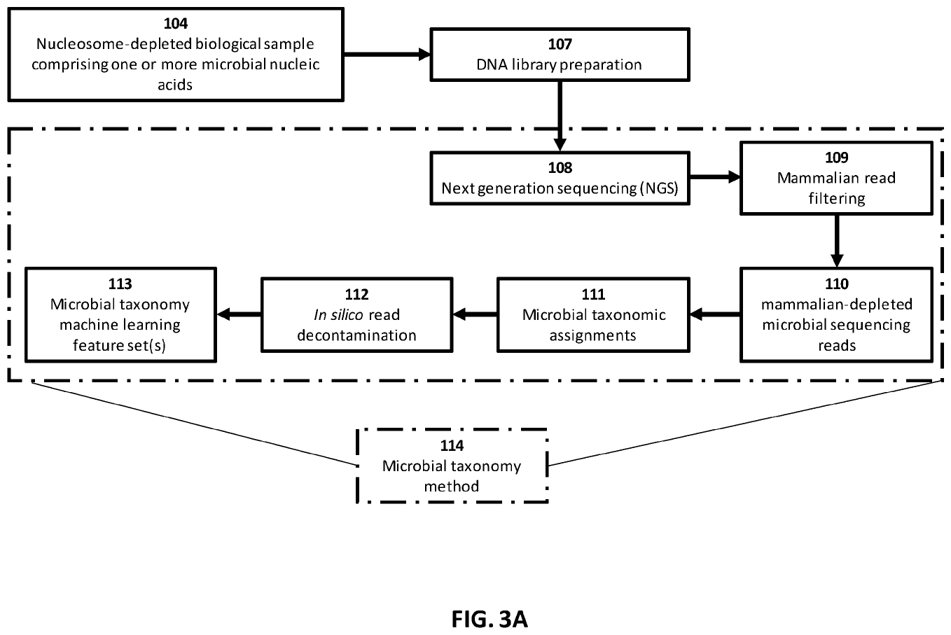

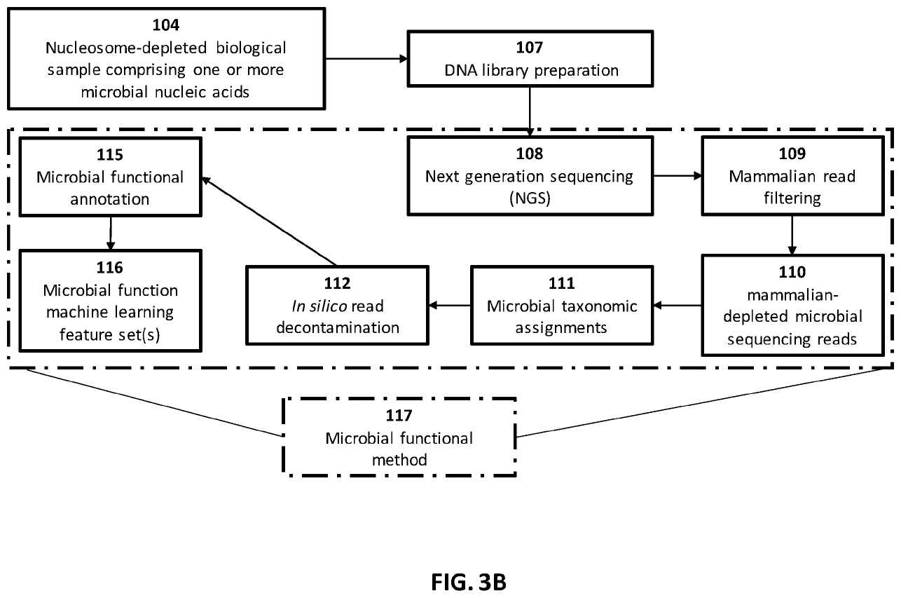

In some cases, the nucleic acid molecule library of enriched microbial nucleic acid molecules of a nucleosome-depleted biological sample 201, described elsewhere herein, may be sequenced and/or analyzed to determine one or more microbial taxonomy features (e.g., microbial abundance and/or distribution of taxonomy of microbes) with a microbial taxonomy workflow and/or method 114, as shown in FIG. 3A. In some cases, the microbial taxonomy method 114 may comprise: sequencing the nucleic acid molecule library, described elsewhere herein, to generate a set of one or more nucleic acid molecule sequencing reads 108; filtering the set of one or more nucleic acid molecule sequencing reads 109 to remove one or more mammalian and/or human nucleic acid molecule sequencing reads thereby generating a set of mammalian-depleted microbial sequencing reads 110; determining microbial taxonomic assignments from the mammalian-depleted microbial sequencing reads 111; and/or decontaminating the microbial taxonomic assignments 112 to generate one or more decontaminated microbial taxonomy feature sets 113. In some cases, filtering may comprise computational mapping the one or more nucleic acid sequencing reads to a human reference genome data to identify and remove one or more mammalian sequencing reads of the one or more nucleic acid molecule sequencing reads. In some cases, decontaminating may comprise in-silico decontamination, experimental control decontamination, or a combination thereof. In some instances, the one or more microbial taxonomy feature sets, labeled with a health state of the subject from which the biological sample was received, obtained, and/or provided from, may be used to train a predictive model, as described elsewhere herein.

In some cases experimental control decontamination may comprise removing and/or subtracting microbial sequencing reads obtained and/or received from a control or blank nucleic acid molecule extraction kit (e.g., a sample collection vessel used to collect and/or provide one or more nucleic acid molecules of a biological sample) from nucleic acid molecule sequencing reads of one or more microbial nucleic acids collected and/or obtained from a biological sample of a subject. In some cases, the control and/or blank nucleic acid molecule extraction kit may have no biological sample introduced into the kit. In some cases, sample of contents of the control and/or blank nucleic acid molecule extraction kit may be obtained and/or collected by swabbing and/or washing the control and/or blank nucleic acid molecule extraction kit and/or vessel with an eluant or a buffer. In some cases, the nucleic acid molecule extraction kit may comprise a kit to extract one or more microbial nucleic acid molecules of a biological sample, as described elsewhere herein. In some cases, removing and/or subtracting a background or noise contaminant nucleic acid molecule sequencing reads(s) (e.g., human and/or microbial nucleic acid molecules) present in a sample collection kit improves classifying, characterizing, and/or diagnostic accuracy, sensitivity, and/or specificity of a model trained with one or more microbial features determined from decontaminated microbial sequencing reads. In some cases, the improvement may comprise at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, or at least about 20% improvement to accuracy, sensitivity, specificity, or any combination thereof performance characteristic of a predictive model, described elsewhere herein.

In some cases, decontamination may comprise removing microbial contaminates from the identified microbial features (i.e., derived from one or more microbial nucleic acid molecules, as described elsewhere herein) prior to training a predictive model (e.g., in-silico decontamination). In some cases, microbes and their corresponding microbial nucleic acids may be removed on the basis of a statistical test, such as a Fisher exact test, that describes differences in presence proportionality of the microbial nucleic acids between negative controls and biological samples. In some cases, a method of experimental control decontamination may comprise the steps of: (i) obtaining one or more negative control vessels (e.g., of a nucleic acid molecule extraction kit) or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic acid molecule reads of the one or more biological samples prior training a predictive model with one or more microbial features of the microbial nucleic acid molecule reads.

In some cases, a nucleic acid molecule library 107 may be generated and/or prepared from the one or more enriched and/or amplified (described elsewhere herein) microbial nucleic acid molecules 104, where the nucleic acid molecule library 107 may be sequenced 108 (e.g., shot gun sequencing, next generation sequencing, and/or sequencing-by-synthesis), as described elsewhere herein. In some cases, the nucleic acid molecule library may comprise a single-stranded DNA nucleic acid molecule library.

In some instances, the nucleic acid molecule library of enriched microbial nucleic acid molecules of a nucleosome-depleted biological sample 201, described elsewhere herein, may be sequenced and/or analyzed to determine one or more microbial function features with a microbial functional workflow and/or method 117, as shown in FIG. 3B. In some cases, the microbial functional workflow and/or method 117 may comprise: sequencing the nucleic acid molecule library to generate a set of one or more nucleic acid molecule sequencing reads 108; filtering the set of one or more nucleic acid molecule sequencing reads 109 to remove one or more mammalian and/or human nucleic acid molecule sequencing reads thereby generating a set of mammalian-depleted microbial sequencing reads 110; determining microbial taxonomic assignments from the mammalian-depleted microbial sequencing reads 111; decontaminating the microbial taxonomic assignments 112; and/or determining one or more microbial functional annotations from of the microbial taxonomic assignments to generate one or more microbial functional feature sets 115. In some cases, filtering may comprise computational mapping the one or more nucleic acid sequencing reads to a human reference genome data to identify and remove one or more mammalian sequencing reads of the one or more nucleic acid molecule sequencing reads. In some cases, decontaminating may comprise in-silico decontamination. In some instances, the one or more microbial functional feature sets, labeled with a health state of the subject from which the biological sample was received, obtained, and/or provided from, may be used to train a predictive model, as described elsewhere herein.

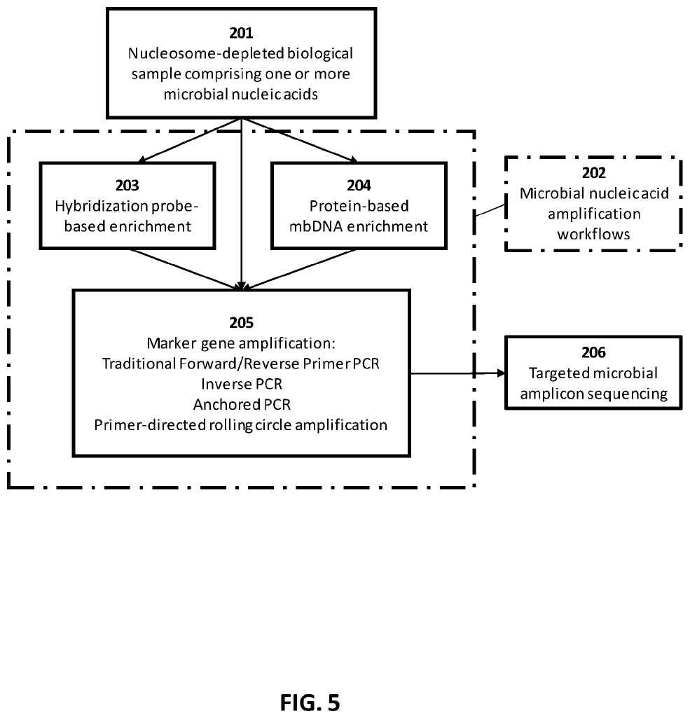

In some embodiments, the biological samples depleted of human nucleic acid molecules (e.g., human nucleic acid molecules coupled to nucleosomes) and/or enriched with microbial nucleic acid molecules 201 may be enriched and amplified with one or more microbial nucleic acid amplification workflows 202 as shown in FIG. 5. In some cases, the microbial nucleic acid amplification workflows 202 may comprise: hybridization probe enrichment 203, protein microbial DNA enrichment 204, or a combination thereof. In some cases, the enriched microbial nucleic acid molecules may then be amplified by one or more marker gene amplification methods 205. In some cases, the amplified and/or further enriched biological sample after enrichment and/or amplification may be sequenced by target microbial amplicon sequencing (206), may comprise shotgun sequencing, next generation sequencing, sequencing by synthesis, or a combination thereof. In some instances, marker gene amplification 205 may comprise forward and/or reverse primer polymerase chain reaction (PCR), inverse PCR, anchored PCR, primer-directed rolling circle amplification, or any combination thereof.

In some cases, hybridization probe enrichment may comprise exposing, providing, and/or incubating one or more hybridization probes with the biological sample, where the one or more hybridization probes are configured to bind to non-mammalian nucleic acid molecules e.g., microbial DNA, RNA, cell-free DNA, cell-free RNA, or any combination thereof. In some cases, the one or more hybridization probes may comprise nucleic acid molecules. In some cases, the one or more nucleic acid molecule hybridization probes may comprise a sequence configured to hybridize to microbial nucleic acid molecule genomic features. In some cases, the microbial nucleic acid molecule genomic features may comprise one or more microbial genes or a portions thereof. In some cases, the microbial nucleic acid molecule genomic feature may comprise: ribosomal RNA gene 5S; ribosomal RNA gene 16S; ribosomal RNA gene 23S; bacterial house keeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof. In some cases, the microbial marker genes may comprise one or more fungal genes: ribosomal RNA gene 18S, ribosomal RNA gene 5.8S, ribosomal RNA gene 28S, and the internal transcribed spacer regions 1 and 2.

In some instances, the method of enriching microbial nucleic acids by hybridization probes may comprise: exposing, providing, and/or combining a nucleosome-depleted biological sample with one or more hybridization probes; incubating the hybridization probe and nucleosome-depleted biological sample under conditions to promote nucleic acid base pair (i.e., nucleic acid base hybridization) between the hybridization probe and one or more microbial nucleic acid molecules of the biological sample; separating and/or removing the unbound hybridization probes and hybridization probes bound to the one or more microbial nucleic acid molecules of the biological sample, thereby enriching the one or more microbial nucleic acid molecules of the biological sample. In some instances, the method may further comprise washing the hybridization probes bound to the one or more microbial nucleic acid molecules. In some instances, washing may be configured to remove non-specifically associated nucleic acid molecules and other reaction components that may couple, hybridize, and/or bind to the hybridization probes.

In some cases, the nucleosome depleted biological sample, described elsewhere herein, may be enriched by protein-based enrichment 204 configured to enrich one or more microbial nucleic acid molecules of the biological sample. In some cases, a method of protein-based enrichment may comprise: exposing, providing, and/or combining a nucleosome-depleted biological sample with one or more recombinant CXXC-domain proteins to form a protein-binding reaction; incubating the protein-DNA binding reaction under conditions that promote an interaction between the recombinant CXXC-domain proteins and non-methylated CpG motifs of the one or more microbial nucleic acid molecules of the nucleosome depleted biological sample; separating unbound recombinant CXXC-domain proteins and recombinant CXXC-domain proteins bound to the non-methylated CpG nucleic acid fragments from the remainder of the protein-DNA binding reaction, thereby enriching the one or more microbial nucleic acid molecules. In some cases, the method of protein-based enrichment may further comprise washing the recombinant CXXC-domain proteins bound to the non-methylated CpG nucleic acid fragments to remove non-specifically associated nucleic acid molecules and the remainder of protein-DNA binding reaction components.

In some instances, marker gene amplification may generate one or more microbial nucleic acid molecule amplicons. In some cases, the one or more microbial nucleic acid molecule amplicons may comprise one or more genomic features. The one or more genomic features may comprise microbial phylogenetic marker genes or marker gene fragments thereof. In some cases, the microbial phylogenetic marker genes may comprise bacterial, fungal, or any combination thereof marker genes. The microbial phylogenic marker genes may comprise bacterial marker genes or marker fragments thereof. The microbial marker genes may comprise fungal marker genes or marker gene fragments thereof. In some cases, the bacterial marker genes may comprise: ribosomal RNA gene 5S; ribosomal RNA gene 16S; ribosomal RNA gene 23S; bacterial house keeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some cases, the fungal marker genes may comprise one or more of: ribosomal RNA gene 18S, ribosomal RNA gene 5.8S, ribosomal RNA gene 28S, and the internal transcribed spacer regions 1 and 2.

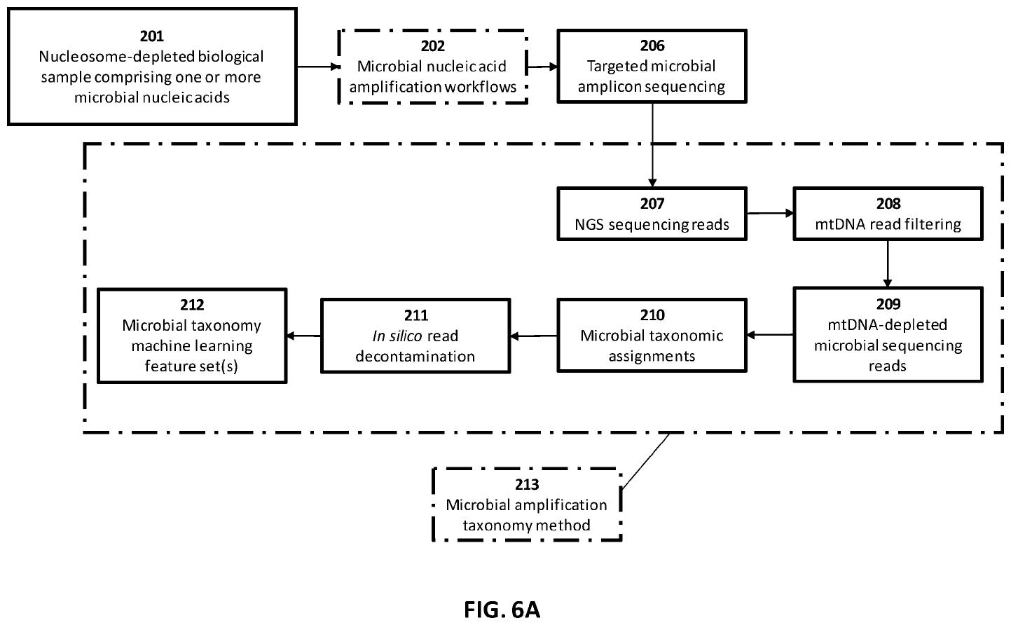

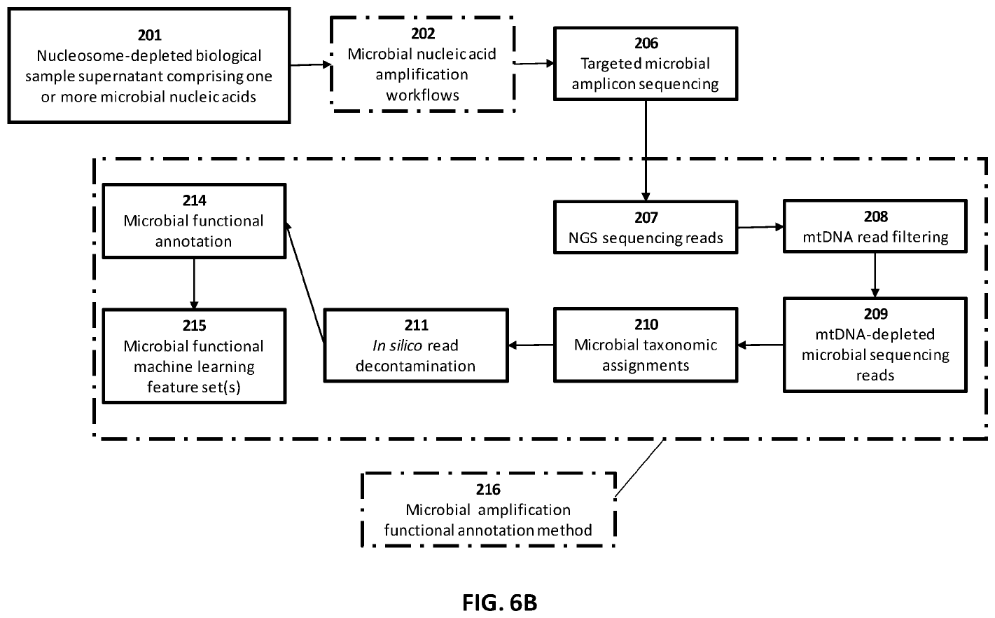

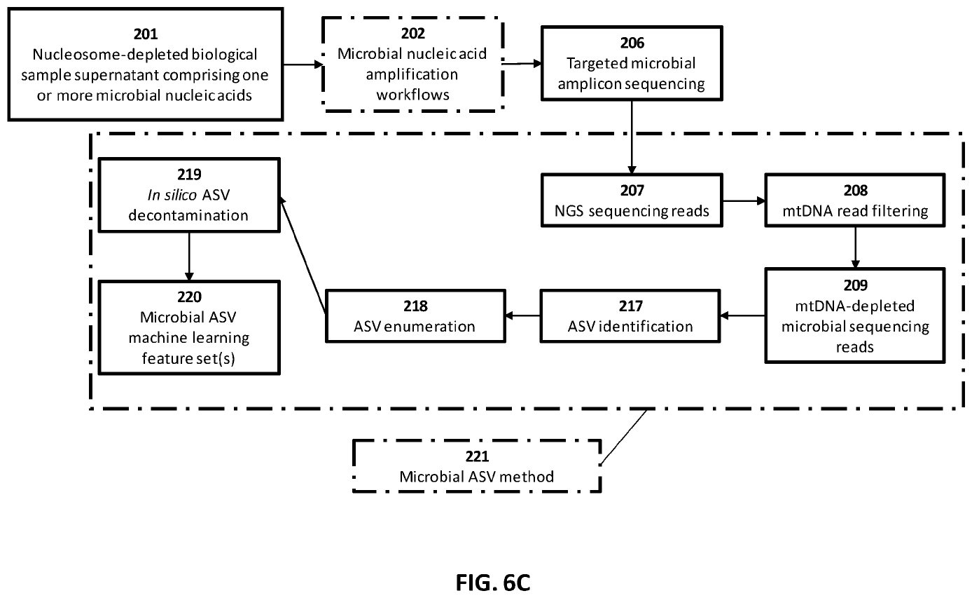

In some cases, the enriched microbial nucleic acid molecules of the nucleosome depleted biological sample 201 may be amplified by one or more microbial nucleic acid amplification workflows 202 and analyzed and/or processed by a workflow and/or method 213 to generate one or more microbial taxonomy feature sets 212 as seen in FIG. 6A. In some cases, the workflow and/or method may comprise: sequencing the one or more microbial amplicons produced by amplification (206), described elsewhere herein, thereby generating one or more sets of microbial sequencing reads (207); filtering the one or more sets of microbial sequencing reads to remove one or more microbial mitochondrial nucleic acid molecules (e.g., microbial mitochondrial DNA) 208 to generate one or more sets of microbial mitochondrial depleted microbial nucleic acid sequencing reads 209; assigning and/or determining microbial taxonomy of the one or more sets of microbial mitochondrial depleted microbial nucleic acid sequencing reads 210; decontaminating the assigned and/or determine microbial taxonomy 211; determining microbial functional annotations of the decontaminated microbial taxonomy 214; and/or to produce one or more microbial functional feature sets 215. In some cases, filtering the one or more sets of microbial sequencing reads to remove one or more microbial mitochondrial nucleic acid molecules may comprise computational mapping the one or more microbial nucleic acid sequencing reads to a mitochondrial reference genome data to identify and remove one or more mitochondrial DNA sequencing reads. In some cases, decontaminating may comprise in-silico decontamination. In some instances, the one or more microbial taxonomy feature sets, labeled with a health state of the subject from which the biological sample was received, obtained, and/or provided from, may be used to train a predictive model, as described elsewhere herein.

In some cases, the enriched microbial nucleic acid molecules of the nucleosome depleted biological sample 201 may be amplified by one or more microbial nucleic acid amplification workflows 202 and analyzed and/or processed by a workflow and/or method 216 to generate one or more microbial functional feature sets 215 as seen in FIG. 6B. In some cases, the workflow and/or method may comprise: sequencing the one or more microbial amplicons produced by amplification (206), described elsewhere herein, thereby generating one or more sets of microbial sequencing reads (207); filtering the one or more sets of microbial sequencing reads to remove one or more microbial mitochondrial nucleic acid molecules (e.g., microbial mitochondrial DNA) 208 to generate one or more sets of microbial mitochondrial depleted microbial nucleic acid sequencing reads 209; assigning and/or determining microbial taxonomy of the one or more sets of microbial mitochondrial depleted microbial nucleic acid sequencing reads 210; decontaminating the assigned and/or determine microbial taxonomy 211 to produce one or more microbial taxonomy feature sets 212. In some cases, filtering the one or more sets of microbial sequencing reads to remove one or more microbial mitochondrial nucleic acid molecules may comprise computational mapping the one or more microbial nucleic acid sequencing reads to a mitochondrial reference genome data to identify and remove one or more mitochondrial DNA sequencing reads. In some cases, decontaminating may comprise in-silico decontamination. In some instances, the one or more microbial taxonomy feature sets, labeled with a health state of the subject from which the biological sample was received, obtained, and/or provided from, may be used to train a predictive model, as described elsewhere herein.

In some cases, the enriched microbial nucleic acid molecules of the nucleosome depleted biological sample 201 may be amplified by one or more microbial nucleic acid amplification workflows 202 and analyzed and/or processed by a workflow and/or method 221 to generate one or more microbial amplicon sequence variant (ASV) feature sets 220 as seen in FIG. 6C. In some cases, the workflow and/or method may comprise: sequencing the one or more microbial amplicons produced by amplification (206), described elsewhere herein, thereby generating one or more sets of microbial sequencing reads (207); filtering the one or more sets of microbial sequencing reads to remove one or more microbial mitochondrial nucleic acid molecules (e.g., microbial mitochondrial DNA) 208 to generate one or more sets of microbial mitochondrial depleted microbial nucleic acid sequencing reads 209; identifying, assigning and/or determining ASV features of the one or more sets of microbial mitochondrial depleted microbial nucleic acid sequencing reads 217; enumerating the ASV features 218; decontaminating (e.g., in-silico decontamination, described elsewhere herein) the enumerated ASV features 219 to produce one or more decontaminated microbial ASV feature sets 220. In some cases, identifying the ASV features of the one or more sets of microbial mitochondrial depleted nucleic acid sequencing reads may comprise identifying mutations and/or single nucleotide polymorphisms of one or more microbial genes. In some cases, sequence variance resulting in mutations and/or single nucleotide polymorphisms of the one or more microbial genes may provide a measure of microbial diversity of a biological sample of a subject. In some cases, the microbial diversity may be utilized to determine one or more microbial features used in training a predictive model, as described elsewhere herein. In some instances, enumerating the ASV features may comprise determining a count or a frequency (e.g., histogram) of a microbial gene variant, mutation, and/or single nucleotide polymorphism of the one or more microbial genes (i.e., the ASV features). In some cases, filtering the one or more sets of microbial sequencing reads to remove one or more microbial mitochondrial nucleic acid molecules may comprise computational mapping the one or more microbial nucleic acid sequencing reads to a mitochondrial reference genome data to identify and remove one or more mitochondrial DNA sequencing reads. In some cases, decontaminating may comprise in-silico decontamination. In some instances, the one or more microbial ASV feature sets, labeled with a health state of the subject from which the biological sample was received, obtained, and/or provided from, may be used to train a predictive model, as described elsewhere herein.

### Predictive Models

The methods and systems described herein utilize or access external capabilities of artificial intelligence, predictive models, and/or machine learning trained on one or more microbial nucleic acid features, e.g., one or more sets of microbial taxonomy features, microbial functional features, microbial ASV features, or any combination thereof, that may classify, diagnose, and/or characterize a health state of a subject, a plurality of subjects and/or one or more groups of subjects. In some cases, the one or more microbial nucleic acid molecule features (e.g., a microbial functional feature, a microbial taxonomic features, etc.), as described elsewhere herein may predict, classify, and/or identify a cancer and/or a non-cancerous disease of one or more subjects. In some cases, one or more microbial nucleic acid molecule features may be used to train one or more predictive models, described elsewhere herein. These trained predictive models may be used to accurately predict, classify, and/or characterize a health state e.g., cancer, non-cancerous diseases, disorders, or any combination thereof, of a subject, a plurality of subjects and/or one or more groups of subjects. Using such a predictive capability, health care providers (e.g., physicians) may make informed, accurate risk-based decisions, thereby improving quality of care and monitoring provided to subjects with cancer, non-cancerous diseases, disorders, or any combination thereof.

The methods and systems of the present disclosure may analyze the presence and/or abundance of a microbes (e.g., abundance of microbes of a particular genus, taxonomy, microbial functional pathways). The presence and/or abundance of microbes may then be used to determine one or more nucleic acid molecule features e.g., non-mammalian and/or microbial nucleic acid molecule features that may predict cancer and/or non-cancerous diseases of one or more subjects. In some cases, the methods, and/or systems, described elsewhere herein, may train a predictive model with the one or more nucleic acid molecule features indicative of a health state e.g., cancer and/or a non-cancerous disease of a subject. In some cases, the trained predictive model may then be used to generate a likelihood (e.g., a prediction) of cancer and/or a non-cancerous disease of one or more subjects that differ from the one or more subjects utilized to train the predictive model. The trained predictive model may comprise an artificial intelligence-based model, such as a machine learning based classifier, configured to process one or more nucleic acid molecule features from the one or more nucleic acid molecules and/or enriched, filtered, and/or amplified one or more nucleic acid molecules, to generate the likelihood of the subject(s) having cancer, a non-cancerous disease, or a disorder. The model may be trained using abundance of microbial taxonomic features or microbial functional pathways from one or more cohorts of subjects, e.g., cancer subjects, subjects with non-cancerous diseases, subjects with no disease and no cancer, cancer subjects receiving a treatment for a cancer, subjects receiving treatment for a non-cancerous disease, or any combination thereof. In some cases, the predictive model may be trained to provide a treatment prediction to treat a cancer of one or more subjects that are not part of the training dataset of the predictive model. Such a predictive model may output a treatment recommendation for the one or more subjects that are not part of the training dataset when provided an input of the patient's presence and abundance of one or more microbes of a hybridization enriched biological sample.

In some embodiments, the disclosure provides methods and/or systems to generate one or more classifiers from one or more subjects' microbial nucleic acid features, described elsewhere herein, identified and/or determined from the one or more subjects' nucleosome-depleted biological samples.

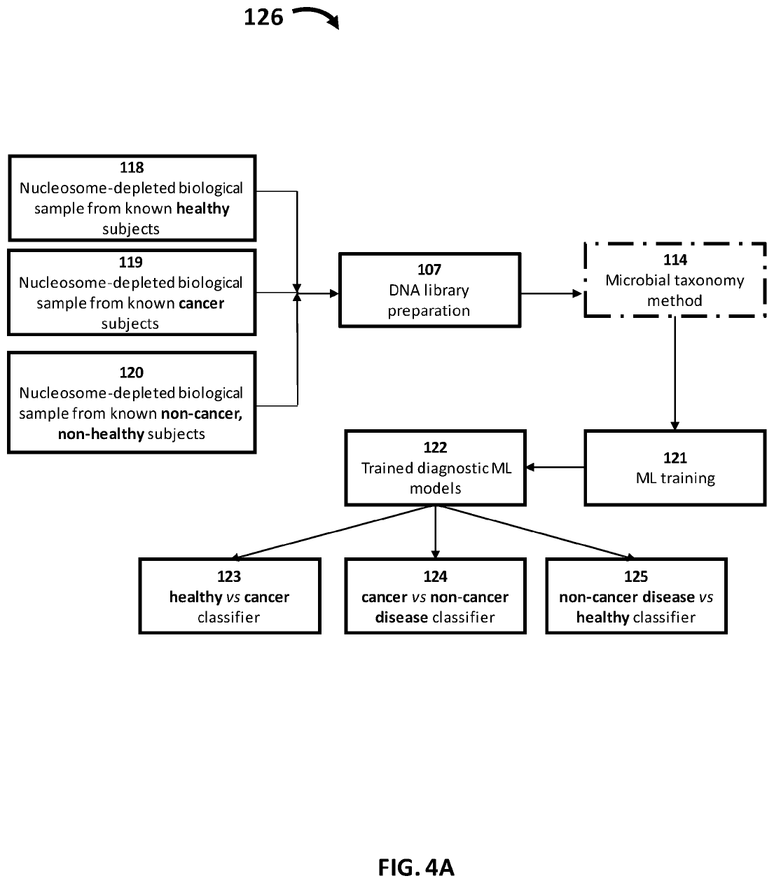

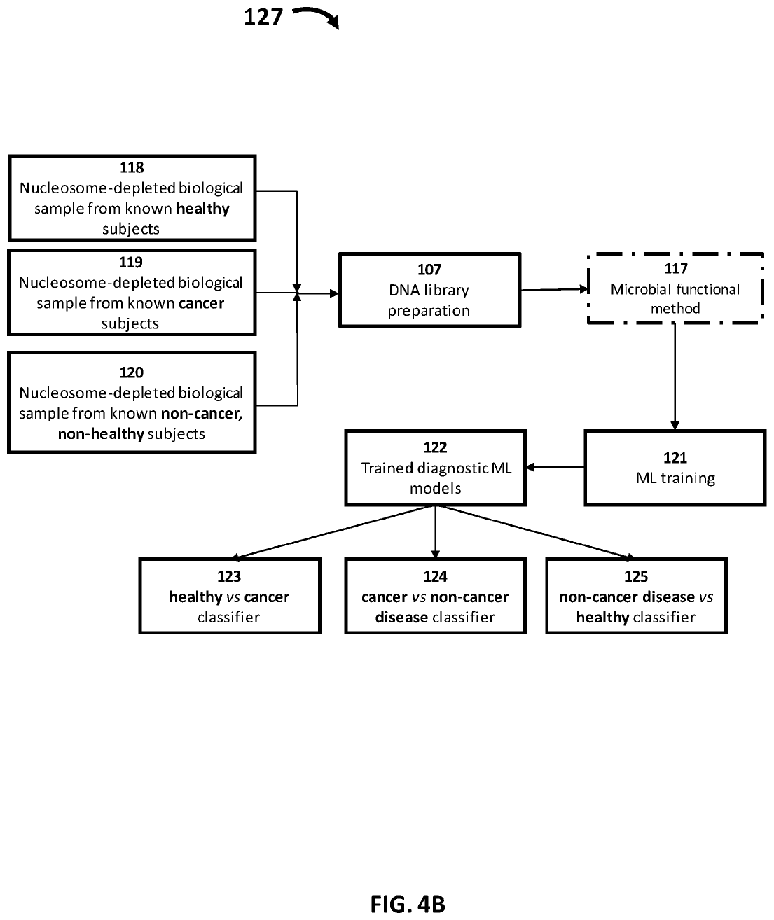

In some cases, the methods and/or systems may comprise training a predictive model with one or more microbial taxonomic features identified and/or determined from one or more subjects' nucleosome-depleted biological samples 126, as shown in FIG. 4A. In some instances, the method may comprise providing, receiving, and/or obtaining nucleosome depleted biological samples, described elsewhere herein, from one or more subjects classified and/or characterized (e.g., by gold standard diagnosis and/or classification methods) as healthy 118, having cancer 119, and/or having a non-cancerous disease 120; generating a nucleic acid molecule (e.g., DNA) sequencing library from one or more microbial nucleic acids of the depleted biological samples 107; sequencing and/or analyzing the one or more microbial nucleic acid molecules with the microbial taxonomy method and/or workflow 114, described elsewhere herein; training a predictive model (e.g., a machine learning classifier) with the one or more microbial taxonomic features determined, identified, and/or analyzed from the microbial nucleic acid molecule sequencing reads 121 thereby generating a trained predictive (e.g., diagnostic) model 122 which comprises a healthy vs. cancer classifier 123, cancer vs. non-cancer disease classifier 124, non-cancer disease vs. healthy classifier 125, or any combination thereof classifiers. In some cases, the trained predictive model and/or the one or more classifiers (123, 124, 125) may be used to screen, diagnose, determine, a health state of one or more subjects' that are not included in the training of the predictive model by providing one or more subjects' nucleosome-depleted one or more microbial taxonomy features. In some cases, the one or more microbial taxonomy features of one or more subjects' nucleosome-depleted biological samples may be determined by methods and systems described elsewhere herein.

In some cases, the methods and/or systems may comprise training a predictive model with one or more microbial functional features identified and/or determined from one or more subjects' nucleosome-depleted biological samples 127, as shown in FIG. 4B. In some instances, the method may comprise providing, receiving, and/or obtaining nucleosome depleted biological samples, described elsewhere herein, from one or more subjects classified and/or characterized (e.g., by gold standard diagnosis and/or classification methods) as healthy 118, having cancer 119, and/or having a non-cancerous disease 120; generating a nucleic acid molecule (e.g., DNA) sequencing library from one or more microbial nucleic acids of the depleted biological samples 107; sequencing and/or analyzing the one or more microbial nucleic acid molecules with the microbial functional method and/or workflow 117, described elsewhere herein; training a predictive model (e.g., a machine learning classifier) with the one or more microbial functional features determined, identified, and/or analyzed from the microbial nucleic acid molecule sequencing reads 121 thereby generating a trained predictive (e.g., diagnostic) model 122 which comprises a healthy vs. cancer classifier 123, cancer vs. non-cancer disease classifier 124, non-cancer disease vs. healthy classifier 125, or any combination thereof classifiers. In some cases, the trained predictive model and/or the one or more classifiers (123, 124, 125) may be used to screen, diagnose, determine, a health state of one or more subjects' that are not included in the training of the predictive model by providing one or more subjects' nucleosome-depleted one or more microbial functional features. In some cases, the one or more microbial functional features of one or more subjects' nucleosome-depleted biological samples may be determined by methods and systems described elsewhere herein.

The predictive model and/or trained predictive model may comprise one or more predictive models. The model may comprise one or more machine learning algorithms. Examples of machine learning algorithms may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, linear regression, k-nearest neighbors, k-means, decision tree, logistic regression, or any combination thereof. The model may be used for classification or regression. The model may likewise involve the estimation of ensemble models, comprised of multiple predictive models, and utilize techniques such as gradient boosting, for example in the construction of gradient-boosting decision trees. The model may be trained using one or more training datasets comprising one or more nucleic acid molecule features, subject data e.g., subject medical history, subject's family medical history, subject vitals (e.g., blood pressure, pulse, temperature, oxygen saturation), subject's known health state, or any combination thereof.

The predictive model may comprise any number of machine learning algorithms. In some embodiments, the random forest machine learning algorithm may be an ensemble of bagged decision trees. The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the machine learning algorithms may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting cancer, non-cancerous disease, disorder, or any combination thereof. The feature may be more relevant for detecting cancer, non-cancerous disease, and/or disorders, if the feature is classified more often than another feature in determining cancer, non-cancerous disease, and/or disorders. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some cases, the machine learning algorithm may prioritize certain features to reduce calculation costs, save processing power, save processing time, increase reliability, or decrease random access memory usage, etc.

Training datasets may be generated from, for example, one or more cohorts of subjects having common cancer, non-cancerous disease, or disorder diagnosis. Training datasets may comprise one or more nucleic acid molecule features in the form of abundance taxonomic assignment features of microbes present in the biological sample and/or microbial functional pathways features of the microbes present in the biological sample of one or more subjects. Features may comprise a corresponding cancer diagnosis of one or more subjects to microbial features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, and/or prognosis of cancer, non-cancerous disease, disorder, or a combination thereof, in the subject (e.g., patient). Clinical outcomes may comprise treatment efficacy (e.g., whether a subject is a positive or a negative responder to a cancer and/or disease-based treatment).

Input features may be structured by aggregating the data into bins or alternatively using a one-hot encoding. Inputs may also include feature values or vectors derived from the previously mentioned inputs, such as cross-correlations.

Training datasets may be constructed from presence and/or abundance of one or more nucleic acid mole features of e.g., one or more microbial taxonomic features, one or more microbial functional pathways, or a combination thereof, identified and/or classified from the enriched and/or amplified nucleic acid molecules of a biological sample indicative of cancer, non-cancerous diseases, disorders, or any combination thereof.

The model may process the input features to generate output values comprising one or more classifications, one or more predictions, or a combination thereof. For example, such classifications or predictions may include a binary classification of a cancer or no cancer present; presence of a non-cancerous disease; presence of a disorder; or any combination thereof classifications of a subject. In some cases, the one or more predictive models and/or machine learning algorithms may classify subjects between a group of categorical labels (e.g., ‘no cancer, non-cancer disease and/or disorder’, ‘apparent cancer, non-cancer disease and/or disorder’, and ‘likely cancer, non-cancer disease and/or disorder’); a likelihood (e.g., relative likelihood or probability) of developing a particular cancer, non-cancerous disease, and/or disorder; a score indicative of a presence of cancer, non-cancer disease and/or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of the model.

In order to train the model (e.g., by determining weights and correlations of the model) to generate real-time classifications or predictions, the model can be trained using training data sets and/or one or more training features, described elsewhere herein. Such datasets and/or features may be sufficiently large to generate statistically significant classifications or predictions. For example, datasets may comprise one or more nucleic acid molecule features derived from sequencing data from fungal, viral, archaeal, bacterial, or any combination thereof microbe presence and/or abundance in one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered, or batch corrected to remove or mitigate confounding factors. For example, within a database, a subset of subjects may be excluded.

The model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features, as described elsewhere herein, e.g., one or more nucleic acid molecule features, vital measurements, subject medical history, subject demographics, or any combination thereof. Neural network techniques, such as dropout or regularization, may be used during training the model to prevent overfitting. The neural network may comprise a plurality of sub-networks, each of which is configured to generate a classification or prediction of a different type of output information, which may be combined to form an overall output of the neural network. The machine learning model may alternatively utilize statistical or related algorithms including random forest, classification and regression trees, support vector machines, discriminant analyses, regression techniques, as well as ensemble and gradient-boosted variations thereof.

When the model generates a classification or a prediction of cancer, non-cancerous disease, disorder, or a combination thereof, a notification (e.g., alert or alarm) may be generated and transmitted to a health care provider, such as a physician, nurse, or other member of the subject's treating team within a hospital. Notifications may be transmitted via an automated phone call, a short message service (SMS), multimedia message service (MMS) message, an e-mail, and/or an alert within a dashboard. The notification may comprise output information such as a prediction of cancer, non-cancerous disease, and/or disorder; a likelihood of the predicted cancer, non-cancerous disease and/or disorder, a time until an expected onset of the cancer, non-cancerous disease and/or disorder; a confidence interval of the likelihood or time, a recommended course of treatment for the cancer, non-cancerous disease and/or disorder, or any combination thereof information.

To validate the performance of the model, different performance metrics may be generated. For example, an area under the receiver-operating characteristic curve (AUROC) may be used to determine the diagnostic, prognostic, screening, or any combination thereof capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the cancer, non-cancerous disease and/or disorder). A “true positive” may refer to an outcome in which positive outcome or result has been correctly generated, when the patient has the cancer, non-cancerous disease and/or disorder (e.g., the patient shows symptoms of the cancer, non-cancerous disease and/or disorder, or the patient's record indicates the cancer, non-cancerous disease and/or disorder). A “false negative” may refer to an outcome in which a negative outcome or result has been generated, but the patient has the cancer, non-cancerous disease and/or disorder (e.g., the patient shows symptoms of the cancer, non-cancerous disease and/or disorder, or the patient's record indicates the cancer, non-cancerous disease and/or disorder). A “true negative” may refer to an outcome in which a negative outcome or result has been generated (e.g., before the actual onset of, or without any onset of, the cancer, non-cancerous disease and/or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a cancer, non-cancerous disease and/or disorder in the subject. As another example, the diagnostic accuracy measure may correspond to prediction of a likelihood of deterioration or recurrence of a cancer, non-cancerous disease and/or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a cancer, non-cancerous disease and/or disorder.

For example, such a pre-determined condition may be that the sensitivity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the cancer, non-cancerous disease and/or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the cancer, non-cancerous disease and/or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training has a diagnostic status indicating that they have either been diagnosed with the biological condition or have not been diagnosed with the cancer, non-cancerous disease and/or disorder.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi: 10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N. Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N. Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5, which is hereby incorporated by reference in its entirety. In some embodiments, ensemble modeling techniques are used; these ensemble modeling techniques are described in the implementation of classification models herein and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Computer Systems

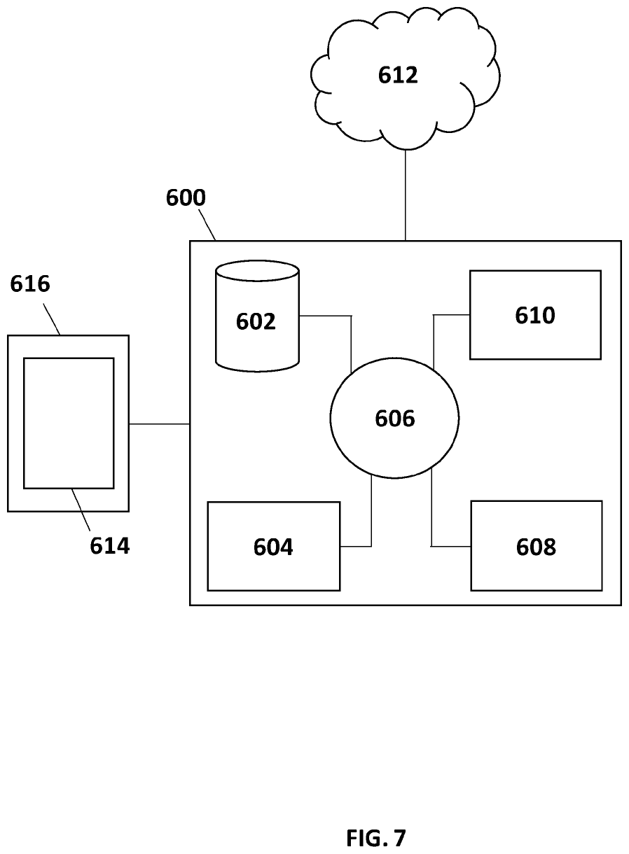

The present disclosure provides computer systems that are programmed to implement methods of the disclosure. FIG. 7 shows a computer system 600 that is programmed or otherwise configured to predict a health state of cancer, non-cancerous disease, or any combination thereof, of one or more subjects; train a predictive model, described elsewhere herein; generate a recommended therapeutic; or any combination thereof methods, described elsewhere herein. The computer system 600 can be an electronic device of a user or a computer system that is remotely located with respect to the electronic device. The electronic device can be a mobile electronic device.

The computer system 600 includes a central processing unit (CPU, also “processor” and “computer processor” herein) 606, which can be a single core or multi core processor, or a plurality of processors for parallel processing. The computer system 600 also includes memory or memory location 604 (e.g., random-access memory, read-only memory, flash memory), electronic storage unit 602 (e.g., hard disk), communication interface 608 (e.g., network adapter) for communicating with one or more other systems, and peripheral devices 610, such as cache, other memory, data storage and/or electronic display adapters. The memory 604, storage unit 602, interface 608 and peripheral devices 610 are in communication with the CPU 606 through a communication bus (solid lines), such as a motherboard. The storage unit 602 can be a data storage unit (or data repository) for storing data. The computer system 600 can be operatively coupled to a computer network (“network”) 612 with the aid of the communication interface 608. The network 612 can be the Internet, an internet and/or extranet, or an intranet and/or extranet that is in communication with the Internet. The network 612 in some cases is a telecommunication and/or data network. The network 612 can include one or more computer servers, which can enable distributed computing, such as cloud computing. The network 612, in some cases with the aid of the computer system 600, can implement a peer-to-peer network, which may enable devices coupled to the computer system 600 to behave as a client or a server.

The CPU 606 can execute a sequence of machine-readable instructions, which can be embodied in a program or software. The instructions may be stored in a memory location, such as the memory 604. The instructions can be directed to the CPU 606, which can subsequently program or otherwise configure the CPU 606 to implement methods of the present disclosure, described elsewhere herein. Examples of operations performed by the CPU 606 can include fetch, decode, execute, and writeback.

The CPU 606 can be part of a circuit, such as an integrated circuit. One or more other components of the system 600 can be included in the circuit. In some cases, the circuit is an application specific integrated circuit (ASIC).

The storage unit 602 can store files, such as drivers, libraries, and saved programs. The storage unit 602 can store user data, e.g., user preferences and user programs. The computer system 600 in some cases can include one or more additional data storage units that are external to the computer system 600, such as located on a remote server that is in communication with the computer system 600 through an intranet or the Internet.

The computer system 600 can communicate with one or more remote computer systems through the network 612. For instance, the computer system 600 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 600 via the network 612.

Methods as described herein can be implemented by way of machine (e.g., computer processor) executable code stored on an electronic storage location of the computer system 600, such as, for example, on the memory 604 or electronic storage unit 602. The machine executable or machine-readable code can be provided in the form of software. During use, the code can be executed by the processor 606. In some cases, the code can be retrieved from the storage unit 602 and stored on the memory 604 for ready access by the processor 606. In some situations, the electronic storage unit 602 can be precluded, and machine-executable instructions are stored on memory 604.

The code can be pre-compiled and configured for use with a machine having a processer adapted to execute the code or can be compiled during runtime. The code can be supplied in a programming language that can be selected to enable the code to execute in a pre-compiled or as-compiled fashion.

In some embodiments, a system, as described elsewhere herein, may comprise a system for diagnosing a cancerous or non-cancerous health state of one or more subjects. In some cases, the system may comprise: (a) one or more processors; and (b) a non-transitory computer readable storage medium including software configured to cause said one or more processors to: (i) receive one or more subjects' one or more nucleic acid molecule sequencing reads of said one or more subjects' biological samples, wherein said one or more nucleic acid molecule sequencing reads comprise a sequence of an amplified one or more genomic features of one or more non-mammalian nucleic acid molecules; and (ii) output a diagnosis of a cancerous or non-cancerous health state of the one or more subjects at least as a result of providing the one or more non-mammalian nucleic acid sequencing reads' one or more genomic features as an input to a trained predictive model.

Aspects of the systems and methods provided herein, such as the computer system 600, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 600 can include or be in communication with an electronic display 616 that comprises a user interface (UI) 614 for providing, for example, a display for visualization of prediction results or an interface for training a predictive model. Examples of UI's include, without limitation, a graphical user interface (GUI) and web-based user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the methods disclosed herein describe and/or show methods with a finite or particular order of steps, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations and the program may comprise program instructions stored on a computer readable memory or programmed steps of the logic circuitry such as the programmable array logic or the field programmable gate array, for example.

The present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

### EXAMPLES

### Example 1: Enrichment and Amplification of Microbial Nucleic Acids Results in Microbial Features that Improve Predictive Model Performance

Biological samples (e.g., a liquid biological sample such as blood) are obtained from subjects of varying clinical classification, namely, subjects that are characterized and/or classified as healthy, having cancer, or having a non-cancerous disease. The biological samples are split into two groups. The first group consists of biological samples from subjects of all the health classifications that are depleted of human or mammalian DNA bound to nucleosomes. The first group of biological samples will also be subjected to further microbial nucleic acid molecule enrichment by use of hybridization probes, protein probes, and/or marker gene amplification, as described elsewhere herein. The second group consists of biological samples from subjects of all health classifications that are not depleted of human or mammalian nucleic acid molecules bound to nucleosomes and are not subjected to further enrichment and/or amplification. Microbial taxonomy and functional features for each group are determined and utilized in combination with the health classification(s) as labels to train two predictive models to classify a health classification based on an input of microbial taxonomy and functional features. Each predictive model is then tested with a set of subjects' data and known health classification to determine accuracy of the model in classifying the subjects' health classification from each subject's microbial taxonomy and functional features determined from each subject's biological sample. The predictive model trained with the microbial taxonomy and functional features determined from depleted, enriched, and amplified microbial nucleic acids of a biological sample outperforms the model trained on microbial taxonomy and functional features that were determined from biological samples not subjected to depletion, enrichment and/or amplification.

The increase in performance between the two models can be understood as an optimization of the microbial taxonomy and functional features determined from the microbial nucleic acid molecules of the biological sample. By depleting the biological sample of human and/or mammalian nucleic acid molecules a larger proportion of the sequencing reads remaining in the biological sample will originate from microbial content that would otherwise be washed out by the large human host nucleic acid molecule background in each biological sample. By enabling a lower level of detection of microbial nucleic acid molecules with the methods and systems described herein, a subtle difference in microbial taxonomy and/or microbial functional features may be revealed that more readily differentiates between microbial compositions of subjects and their corresponding health states.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of generating a microbial metagenomic feature set to diagnose cancer, the method comprising:
(a) providing a plurality of subjects' health states and said plurality of subjects' biological samples, wherein said biological samples comprise mammalian nucleic acid molecules and microbial nucleic acid molecules;
(b) removing said mammalian nucleic acid molecules from said biological samples with an affinity capture reagent;
(c) sequencing said microbial nucleic acid molecules to generate microbial sequencing reads; and
(d) generating said microbial metagenomic feature set to diagnose said cancer by combining a metagenomic feature abundances of said microbial sequencing reads and said plurality of subjects' health states.

2. The method of claim 1, wherein said metagenomic feature set comprises microbial taxonomic abundance.

3. The method of claim 1, wherein said metagenomic feature set comprises computationally inferred microbial biochemical pathways and said microbial biochemical pathways' associated abundances.

4. The method of claim 1, wherein said metagenomic feature set comprises microbial phylogenetic marker genes or marker gene fragments thereof.

5. The method of claim 1, wherein said biological sample comprises a liquid biological sample, and wherein said liquid biological sample comprises: plasma, serum, whole blood, urine, cerebral spinal fluid, saliva, sweat, tears, exhaled breath condensate, or any combination, dilution, or processed fraction thereof.

6. The method of claim 5, wherein (b) comprises:
(a) contacting said liquid biological sample with a solid support comprising immobilized anti-nucleosome antibodies to form antibody-nucleosome interaction complexes;
(b) separating said solid support from said liquid biological sample to concentrate said antibody-nucleosome interaction complexes; and
(c) purifying the remaining one or more nucleosome-depleted microbial nucleic acid molecules.

7. The method of claim 6, wherein said anti-nucleosome antibodies are configured to bind to an epitope comprising DNA and one or more histone proteins.

8. The method of claim 6, wherein said solid supports comprise a magnetic bead, agarose bead, non-magnetic latex, functionalized Sepharose, pH-sensitive polymers or any combination thereof.

9. The method of claim 5, wherein (b) comprises:
(a) contacting said liquid biological sample with one or more anti-nucleosome antibodies to form antibody-nucleosome interaction complexes;
(b) contacting said antibody-nucleosome interaction complexes with a solid support, wherein a surface of said solid support comprises a binding moiety configured to couple to said antibody-nucleosome interaction complex;
(c) separating said solid support from said liquid biological sample to concentrate said antibody-nucleosome interaction complexes; and
(d) purifying the remaining nucleosome-depleted microbial nucleic acid molecules.

10. The method of claim 9, wherein said anti-nucleosome antibodies comprise a plurality of epitope tags.

11. The method of claim 10, wherein said plurality of epitope tags comprise an N- or C-terminal 6×-histidine tag, green fluorescent protein (GFP), myc, hemagglutinin (HA), Fc fusion, biotin or any combination thereof.

12. The method of claim 9, wherein said solid support comprises a magnetic bead, agarose bead, non-magnetic latex, functionalized Sepharose, pH-sensitive polymers or any combination thereof.

13. The method of claim 9, wherein said solid support comprises covalently immobilized affinity agents.

14. The method of claim 13, wherein said affinity reagents comprise streptavidin, antibodies specific for 6×-histidine tag, green fluorescent protein (GFP), myc, hemagglutinin (HA), biotin, or any combination thereof.

15. The method of claim 13, wherein said affinity agents comprise anti-species antibodies.

16. The method of claim 1, wherein (c) comprises:
(a) generating single-stranded DNA libraries from said microbial nucleic acid molecules;
(b) performing shotgun metagenomic sequencing analysis of said single-stranded DNA libraries to produce sequencing reads;
(c) filtering said sequencing reads to produce mammalian DNA-depleted microbial sequencing reads; and
(d) decontaminating said mammalian DNA-depleted microbial sequencing reads to remove non-endogenous microbial sequencing reads.

17. The method of claim 16, wherein said decontaminating comprises in-silico decontamination.

18. The method of claim 16, wherein said filtering comprises computationally mapping said sequencing reads to a human reference genome database.

19. The method of claim 1, wherein (c) comprises:
(a) amplifying genomic features of said microbial nucleic acid molecules, thereby generating amplified genomic features;
(b) sequencing said amplified genomic features to generate sequencing reads;
(c) filtering said sequencing reads to produce mitochondrial DNA-depleted microbial sequencing reads; and
(d) decontaminating said mitochondrial DNA-depleted microbial sequencing reads to remove non-endogenous microbial sequencing reads.

20. The method of claim 19, wherein said decontaminating comprises in-silico decontamination.

21. The method of claim 19, wherein said genomic features comprise microbial phylogenetic marker genes or marker gene fragments thereof.

22. The method of claim 21, wherein said microbial phylogenetic marker genes comprise bacterial marker genes or marker gene fragments thereof.

23. The method of claim 21, wherein said microbial phylogenetic marker genes comprise fungal marker genes or marker gene fragments thereof.

24. The method of claim 22, wherein said bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

25. The method of claim 23, wherein said fungal marker genes comprise one or more of: ribosomal RNA gene 18S, ribosomal RNA gene 5.8S, ribosomal RNA gene 28S, and the internal transcribed spacer regions 1 and 2.

26. The method of claim 21, wherein said microbial phylogenetic marker genes comprise bacterial, fungal, or any combination thereof marker genes.

27. The method of claim 19, wherein amplifying comprises performing a polymerase chain reaction or derivatives thereof.

28. The method of claim 27, wherein said derivatives thereof comprise inverse PCR, anchored PCR, primer-directed rolling circle amplification, or any combination thereof.

29. The method of claim 1, wherein (c) comprises enriching said microbial nucleic acid molecules.

30. The method of claim 29, wherein said enriching comprises:
(a) combining purified nucleosome-depleted microbial nucleic acid molecules with hybridization probes, wherein said hybridization probes comprise a nucleic acid sequence complementarity to microbial genomic features;
(b) incubating said hybridization probes and said nucleosome-depleted microbial nucleic acid molecules under conditions that promote nucleic acid base pairing between target nucleic acid features and said hybridization probes;
(c) separating unbound hybridization probes and hybridized probes bound to said microbial nucleic acid molecules; and
(d) washing said hybridized probes bound to said microbial nucleic acid molecules, thereby generating enriched microbial nucleic acid molecules.

31. The method of claim 30, wherein said washing is to remove non-specifically associated nucleic acid molecules and other reaction components.

32. The method of claim 29, wherein said enriching comprises:
(a) combining purified nucleosome-depleted microbial nucleic acid molecules with recombinant CXXC-domain proteins to form a protein-DNA binding reaction;
(b) incubating said protein-DNA binding reaction under conditions that promote an interaction between said recombinant CXXC-domain proteins and non-methylated CpG motifs of said nucleosome-depleted microbial nucleic acid molecules;
(c) separating unbound recombinant CXXC-domain proteins and recombinant CXXC-domain proteins bound to said non-methylated CpG motifs from a remainder of said protein-DNA binding reaction; and
(d) washing said recombinant CXXC-domain proteins bound to said non-methylated CpG nucleic acid fragments, thereby generating enriched nucleic acid molecules for amplification.

33. The method of claim 32, wherein said washing is configured to remove non-specifically associated nucleic acid molecules and said remainder of protein-DNA binding reaction components.

34. The method of claim 32, wherein said amplification comprises performing a polymerase chain reaction or derivatives thereof.

35. The method of claim 34, wherein said derivatives thereof comprise inverse PCR, anchored PCR, primer-directed rolling circle amplification, or any combination thereof.

36. The method of claim 1, wherein said plurality of subjects comprise human, non-human mammal, or any combination thereof subjects.

37. The method of claim 1, wherein said mammalian nucleic acid molecules comprise DNA, RNA, cell-free DNA, cell-free RNA, exosomal DNA, exosomal RNA, or any combination thereof nucleic acid molecules, and wherein said microbial nucleic acid molecules comprise microbial cell-free RNA, microbial cell-free DNA, microbial RNA, microbial DNA, or any combination thereof nucleic acid molecules.

38. The method of claim 1, wherein said cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

39. The method of claim 1, wherein said cancer comprises a cancer of stage I, II, or III.

40. The method of claim 1, comprising generating a trained predictive model, wherein said trained predictive model is trained with said microbial metagenomic feature set and said health state of said one or more subjects.

41. The method of claim 40, wherein said trained predictive model comprises a machine learning model, one or more machine learning models, an ensemble of machine learning models, or any combination thereof.

42. The method of claim 40, wherein said trained predictive model comprises a regularized machine learning model.

43. The method of claim 41, wherein said machine learning model comprises a machine learning classifier.

44. The method of claim 41, wherein said machine learning model comprises a gradient boosting machine, neural network, support vector machine, k-means, classification trees, random forest, regression, or any combination thereof machine learning models.

45. A method of diagnosing a cancer of a subject, the method comprising:
(a) providing a biological sample of said subject, wherein said biological sample comprises mammalian nucleic acid molecules and microbial nucleic acid molecules;
(b) removing said mammalian nucleic acid molecules from said biological sample with an affinity capture reagent;
(c) sequencing a plurality microbial nucleic acid molecules of said biological sample to generate microbial sequencing reads;
(d) generating metagenomic feature abundances of said microbial sequencing reads; and
(e) outputting said diagnosis of said cancer of said subject at least as a result of providing said microbial metagenomic feature abundances as an input to a trained predictive model.

46. The method of claim 45, wherein said microbial metagenomic feature sets comprise microbial taxonomic abundance.

47. The method of claim 45, wherein said microbial metagenomic feature sets comprise computationally inferred microbial biochemical pathways and their associated abundance.

48. The method of claim 45, wherein said microbial metagenomic feature sets comprise microbial phylogenetic marker genes or marker gene fragments thereof.

49. The method of claim 45, wherein said biological sample comprisesa liquid biological sample, and wherein said liquid biological sample comprises plasma, serum, whole blood, urine, cerebral spinal fluid, saliva, sweat, tears, exhaled breath condensate, or any combination, dilution, or processed fraction thereof.

50. The method of claim 49, wherein (b) comprises:
(a) contacting said liquid biological sample with a solid support comprising immobilized anti-nucleosome antibodies, wherein said anti-nucleosome antibodies are configured to form antibody-nucleosome interaction complexes;
(b) separating said solid support from said liquid biological sample to concentrate said antibody-nucleosome interaction complexes; and
(c) purifying the remaining nucleosome-depleted microbial nucleic acid molecules.

51. The method of claim 50, wherein said anti-nucleosome antibodies recognize an epitope comprising DNA and one or more histone proteins.

52. The method of claim 50, wherein said solid support comprises a magnetic bead, an agarose bead, non-magnetic latex, functionalized Sepharose, pH-sensitive polymers or any combinations thereof.

53. The method of claim 49, wherein (b) comprises:
(a) contacting said liquid biological sample with anti-nucleosome antibodies to form antibody-nucleosome interaction complexes;
(b) contacting said antibody-nucleosome interaction complexes with a solid support configured to bind to said antibody-nucleosome interaction complexes;
(c) separating said solid support from said liquid biological sample to concentrate said antibody-nucleosome interaction complexes; and
(d) purifying the remaining nucleosome-depleted microbial nucleic acids.

54. The method of claim 53, wherein said anti-nucleosome antibodies comprise epitope tags.

55. The method of claim 54, wherein said epitope tags comprise an N- or C-terminal 6×-histidine tag, green fluorescent protein (GFP), myc, hemagglutinin (HA), Fc fusion, biotin, or any combination thereof.

56. The method of claim 53, wherein said solid support comprise a magnetic bead, agarose bead, non-magnetic latex, functionalized Sepharose, pH-sensitive polymers or any combination thereof.

57. The method of claim 53, wherein said solid support comprises covalently immobilized affinity agents.

58. The method of claim 57, wherein said covalently immobilized affinity agents comprise streptavidin, antibodies specific for 6×-histidine tag, green fluorescent protein (GFP), myc, hemagglutinin (HA), biotin, or any combination thereof.

59. The method of claim 57, wherein said covalently immobilized affinity agents comprise anti-species antibodies.

60. The method of claim 45, wherein (c) comprises:
(a) generating single-stranded DNA libraries from said microbial nucleic acid molecules;
(b) performing shotgun metagenomic sequencing analysis of said single-stranded DNA libraries to produce sequencing reads;
(c) filtering said sequencing reads to produce mammalian DNA-depleted microbial sequencing reads; and
(d) decontaminating said mammalian DNA-depleted microbial sequencing reads to remove non-endogenous microbial sequencing reads.

61. The method of claim 60, wherein said decontaminating comprises in-silico decontamination of said mammalian DNA-depleted microbial sequencing reads.

62. The method of claim 60, wherein said filtering comprises computationally mapping said sequencing reads to a human reference genome database.

63. The method of claim 45, wherein (c) comprises:
(a) amplifying genomic features of said microbial nucleic acid molecules, thereby generating amplified genomic features;
(b) sequencing said amplified genomic features to generate sequencing reads;
(c) filtering said sequencing reads to produce mitochondrial DNA-depleted microbial sequencing reads; and
(d) decontaminating said mitochondrial DNA-depleted microbial sequencing reads to remove non-endogenous microbial sequencing reads.

64. The method of claim 63, wherein said decontaminating comprises in-silico decontamination of said mitochondrial DNA-depleted microbial sequencing reads.

65. The method of claim 63, wherein said genomic features comprise microbial phylogenetic marker genes or marker gene fragments thereof.

66. The method of claim 65, wherein said microbial phylogenetic marker genes comprise bacterial marker genes or marker gene fragments thereof.

67. The method of claim 65, wherein said microbial phylogenetic marker genes comprise fungal marker genes or marker gene fragments thereof.

68. The method of claim 66, wherein said bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

69. The method of claim 67, wherein said fungal marker genes comprise one or more of: ribosomal RNA gene 18S, ribosomal RNA gene 5.8S, ribosomal RNA gene 28S, and the internal transcribed spacer regions 1 and 2.

70. The method of claim 65, wherein said microbial phylogenetic marker genes comprise bacterial, fungal, or any combination thereof marker genes.

71. The method of claim 63, wherein said amplifying comprises performing a polymerase chain reaction or derivatives thereof.

72. The method of claim 71, wherein said derivatives thereof comprise inverse PCR, anchored PCR, primer-directed rolling circle amplification, or any combination thereof.

73. The method of claim 45, wherein (c) comprises enriching said microbial nucleic acid molecules.

74. The method of claim 73, wherein said enriching of said microbial nucleic acid molecules comprises:
(a) combining purified nucleosome-depleted microbial nucleic acid molecules with hybridization probes, wherein said hybridization probes comprise a nucleic acid sequence complimentary to microbial genomic nucleic acid features;
(b) incubating said hybridization probes and said nucleosome-depleted microbial nucleic acid molecules under conditions that promote nucleic acid base pairing between said microbial genomic nucleic acid features and said hybridization probes;
(c) separating unbound hybridization probes and hybridized probes bound to said nucleosome-depleted microbial nucleic acid molecules; and
(d) washing said hybridized probes bound to said nucleosome-depleted microbial nucleic acid molecules, thereby generating one or more enriched microbial nucleic acid molecules.

75. The method of claim 74, wherein said washing is configured to remove non-specifically associated nucleic acid molecules and other reaction components.

76. The method of claim 73, wherein said enriching said microbial nucleic acid molecules comprises:
(a) combining purified nucleosome-depleted microbial nucleic acid molecules with recombinant CXXC-domain proteins to form a protein-DNA binding reaction;
(b) incubating said protein-DNA binding reaction under conditions that promote an interaction between said recombinant CXXC-domain proteins and non-methylated CpG motifs of said nucleosome-depleted microbial nucleic acid molecules;
(c) separating unbound recombinant CXXC-domain proteins and recombinant CXXC-domain proteins bound to said non-methylated CpG nucleic acid fragments from a remainder of the protein-DNA binding reaction components;
(d) washing said recombinant CXXC-domain proteins bound to said non-methylated CpG nucleic acid fragments, thereby generating enriched nucleic acid molecules for amplification.

77. The method of claim 76, wherein said washing is configured to remove non-specifically associated nucleic acid molecules and said remainder of said protein-DNA binding reaction components.

78. The method of claim 76, wherein said amplification comprises performing a polymerase chain reaction or derivatives thereof.

79. The method of claim 78, wherein said derivatives thereof comprise inverse PCR, anchored PCR, primer-directed rolling circle amplification, or any combination thereof.

80. The method of claim 45, wherein said subjects comprises a human, non-human mammal, or any combination thereof subject.

81. The method of claim 45, wherein said mammalian nucleic acid molecules comprise DNA, RNA, cell-free RNA, cell-free DNA, exosomal DNA, exosomal RNA, or any combination thereof nucleic acid molecules, and wherein said microbial nucleic acid molecules comprise microbial cell-free DNA, microbial cell-free RNA, microbial DNA, microbial RNA, or any combination thereof nucleic acid molecules.

82. The method of claim 45, wherein said cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof.

83. The method of claim 45, wherein said cancer comprises a cancer of stage I, II, or III.

84. The method of claim 45, wherein said trained predictive model is trained with microbial metagenomic feature sets and corresponding health state of one or more subjects.

85. The method of claim 45, wherein said trained predictive model comprises a machine learning model, one or more machine learning models, an ensemble of machine learning models, or any combination thereof.

86. The method of claim 45, wherein said trained predictive model comprises a regularized machine learning model.

87. The method of claim 86, wherein said machine learning model comprises a machine learning classifier.

88. The method of claim 86, wherein said machine learning model comprises a gradient boosting machine, neural network, support vector machine, k-means, classification trees, random forest, regression, or any combination thereof machine learning models.

89. The method of claim 45, wherein said subject is suspected of having cancer or a disease.

90. The method of claim 45, wherein said subject's imaging results indicate a potential presence of cancer.

91. A system for diagnosing cancer of a subject, the system comprising:
(a) a processor; and
(b) a non-transitory computer readable storage medium including software configured to cause said processor to:
(i) receive a subject's mammalian nucleosome-depleted nucleic acid molecule sequencing reads, wherein said mammalian nucleosome-depleted nucleic acid molecule sequencing reads comprise metagenomic features of microbial nucleic acid molecules; and
(ii) output a diagnosis of said cancer of said subject at least as a result of providing said metagenomic features as an input to a trained predictive model.

92. The system of claim 91, wherein said metagenomic features comprise microbial taxonomic abundance.

93. The system of claim 91, wherein said metagenomic features comprise computationally inferred microbial biochemical pathways and their associated abundance.

94. The system of claim 91, wherein said metagenomic features comprise microbial phylogenetic marker genes or marker gene fragments thereof.

95. The system of claim 91, wherein said mammalian nucleosome-depleted nucleicacid molecule sequencing reads are obtained and/or received from said subjects' liquid biological samples, wherein said liquid biological samples comprise: plasma, serum, whole blood, urine, cerebral spinal fluid, saliva, sweat, tears, exhaled breath condensate, or any combination, dilution, or processed fraction thereof.

96. The system of claim 95, wherein said mammalian nucleosome-depleted nucleic acid molecule sequencing reads are produced by:
(a) contacting said liquid biological sample with a solid support to form antibody-nucleosome interaction complexes, wherein said solid support comprises a surface comprising anti-nucleosome antibodies coupled thereto;
(b) separating said solid support from said liquid biological sample to concentrate said antibody-nucleosome interaction complexes;
(c) purifying said remaining nucleosome-depleted microbial nucleic acid molecules; and
(d) sequencing said purified nucleosome-depleted microbial nucleic acid molecules.

97. The system of claim 96, wherein said anti-nucleosome antibodies are configured to recognize an epitope comprising DNA and histone proteins.

98. The system of claim 96, wherein said solid support comprises a magnetic bead, an agarose bead, non-magnetic latex, functionalized Sepharose, pH-sensitive polymers or any combinations thereof.

99. The system of claim 95, wherein said mammalian nucleosome-depleted nucleic acid molecules' sequencing reads are produced by:
(a) contacting said liquid biological sample with anti-nucleosome antibodies to form antibody-nucleosome interaction complexes;
(b) contacting said antibody-nucleosome interaction complexes with a solid support;
(c) separating said solid support from said liquid biological sample to concentrate said antibody-nucleosome interaction complexes;
(d) purifying the remaining nucleosome-depleted microbial nucleic acid molecules; and
(e) sequencing said purified one or more nucleosome-depleted microbial nucleic acid molecules.

100. The system of claim 99, wherein said anti-nucleosome antibodies comprise epitope tags.

101. The system of claim 100, wherein said epitope tags comprise an N- or C-terminal 6×-histidine tag, green fluorescent protein (GFP), myc, hemagglutinin (HA), Fc fusion, biotin or any combination thereof.

102. The system of claim 99, wherein said solid support comprises a magnetic bead, agarose bead, non-magnetic latex, functionalized Sepharose, pH-sensitive polymers or any combination thereof.

103. The system of claim 99, wherein said solid support comprises covalently immobilized affinity agents.

104. The system of claim 103, wherein said covalently immobilized affinity agents comprise streptavidin, antibodies specific for 6×-histidine tag, green fluorescent protein (GFP), myc, hemagglutinin (HA), biotin, or any combination thereof.

105. The system of claim 103, wherein said covalently immobilized affinity agents comprise anti-species antibodies.

106. The system of claim 91, wherein said mammalian nucleosome-depleted nucleic acid molecule sequencing reads are produced by:
(a) generating single-stranded DNA libraries from said microbial nucleic acid molecules;
(b) performing shotgun metagenomic sequencing analysis of said single-stranded DNA libraries to produce sequencing reads;
(c) filtering said sequencing reads to produce mammalian DNA-depleted microbial sequencing reads; and
(d) decontaminating said mammalian DNA-depleted microbial sequencing reads to remove non-endogenous microbial sequencing reads.

107. The system of claim 106, wherein said decontaminating comprises in-silico decontamination of said mammalian DNA-depleted microbial sequencing reads.

108. The system of claim 106, wherein said filtering comprises computationally mapping said sequencing reads to a human reference genome database.

109. The system of claim 91, wherein said mammalian nucleosome-depleted nucleic acid molecule sequencing reads are produced by:
(a) amplifying genomic features of said microbial nucleic acid molecules, thereby generating amplified genomic features;
(b) sequencing said amplified genomic features to generate sequencing reads;
(c) filtering said sequencing reads to produce mitochondrial DNA-depleted microbial sequencing reads; and
(d) decontaminating said mitochondrial DNA-depleted microbial sequencing reads to remove non-endogenous microbial sequencing reads.

110. The system of claim 109, wherein said decontaminating comprises in-silico decontamination of said mitochondrial DNA-depleted microbial sequencing reads.

111. The system of claim 109, wherein said genomic featurescomprise microbial phylogenetic marker genes or marker gene fragments thereof.

112. The system of claim 111, wherein said microbial phylogenetic marker genes comprise bacterial marker genes or marker gene fragments thereof.

113. The system of claim 111, wherein said microbial phylogenetic marker genes comprise fungal marker genes or marker gene fragments thereof.

114. The system of claim 112, wherein said bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

115. The system of claim 113, wherein said fungal marker genes comprise one or more of: ribosomal RNA gene 18S, ribosomal RNA gene 5.8S, ribosomal RNA gene 28S, and the internal transcribed spacer regions 1 and 2.

116. The system of claim 111, wherein said microbial phylogenetic marker genes comprise bacterial, fungal, or any combination thereof marker genes.

117. The system of claim 109, wherein said amplifying comprises performing a polymerase chain reaction (PCR) or derivatives thereof.

118. The system of claim 117, wherein said derivatives thereof comprise inverse PCR, anchored PCR, primer-directed rolling circle amplification, or any combination thereof.

119. The system of claim 91, wherein said microbial nucleic acid molecules are enriched from said mammalian nucleosome-depleted nucleic acid molecules.

120. The system of claim 119, wherein said enriching of said microbial nucleic acid molecules comprises:
(a) combining purified nucleosome-depleted microbial nucleic acid molecules with hybridization probes, wherein said hybridization probes comprise a nucleic acid sequence complementarity to microbial genomic nucleic acid features;
(b) incubating said hybridization probes and nucleosome-depleted microbial nucleic acid molecules under conditions that promote nucleic acid base pairing between said microbial genomic nucleic acid features and said hybridization probes;
(c) separating unbound hybridization probes and hybridized probes bound to said nucleosome-depleted microbial nucleic acid molecules; and
(d) washing said hybridized probes bound to said nucleosome-depleted microbial nucleic acid molecules, thereby generating enriched microbial nucleic acid molecules.

121. The system of claim 120, wherein said washing is configured to remove non-specifically associated nucleic acid molecules and other reaction components.

122. The system of claim 119, wherein said enriching of said microbial nucleic acid molecules comprises:
(a) combining purified nucleosome-depleted microbial nucleic acid molecules with recombinant CXXC-domain proteins to form a protein-DNA binding reaction;
(b) incubating said protein-DNA binding reaction under conditions that promote an interaction between said recombinant CXXC-domain proteins and non-methylated CpG motifs of said nucleosome-depleted microbial nucleic acid molecules;
(c) separating unbound recombinant CXXC-domain proteins and recombinant CXXC-domain proteins bound to said non-methylated CpG nucleic acid fragments from a remainder of the protein-DNA binding reaction components; and
(d) washing said recombinant CXXC-domain proteins bound to said non-methylated CpG nucleic acid fragments, thereby generating enriched nucleic acid molecules for amplification.

123. The system of claim 122, wherein said washing is configured to remove non-specifically associated nucleic acid molecules and said remainder of said protein-DNA binding reaction components.

124. The system of claim 122, wherein said amplification comprises performing a polymerase chain reaction (PCR) or derivatives thereof.

125. The system of claim 124, wherein said derivatives thereof comprise inverse PCR, anchored PCR, primer-directed rolling circle amplification, or any combination thereof.

126. The system of claim 91, wherein said subject comprises human, non-human mammal, or any combination thereof subjects.

127. The system of claim 91, wherein said mammalian nucleosome-depleted nucleic acid molecule sequencing reads are obtained from mammalian nucleosome-depleted nucleic acid molecules of said subject's biological sample, wherein said biological comprises mammalian nucleic acid molecules comprising DNA, RNA, cell-free RNA, cell-free DNA, exosomal DNA, exosomal RNA, or any combination thereof, and wherein said microbial nucleic acid molecules comprise microbial cell-free DNA, microbial cell-free RNA, microbial DNA, microbial RNA, or any combination thereof.

128. The system of claim 91, wherein said cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma or any combination thereof.

129. The system of claim 91, wherein said cancer comprises a cancer of stage I, II, or III.

130. The system of claim 91, wherein said trained predictive model is trained with metagenomic features and corresponding health states of a plurality of subjects.

131. The system of claim 91, wherein said trained predictive model comprises a machine learning model, one or more machine learning models, an ensemble of machine learning models, or any combination thereof.

132. The system of claim 91, wherein said trained predictive model comprises a regularized machine learning model.

133. The system of claim 131, wherein said machine learning model comprises a machine learning classifier.

134. The system of claim 131, wherein said machine learning model comprises a gradient boosting machine, neural network, support vector machine, k-means, classification trees, random forest, regression, or any combination thereof machine learning models.

135. The system of claim 91, wherein said subject is suspected of having cancer or a disease.

136. The system of claim 91, wherein said subject's imaging results indicate a potential presence of cancer.

137. A method of generating metagenomic features of a sample of cell-free microbial nucleic acid molecules to diagnose a non-oncologic disease, comprising:
(a) contacting said sample of cell-free nucleic acid molecules with a probe, wherein said probe comprises a binding moiety configured to bind to human nucleic acid molecules complexed to proteins;
(b) removing said probe bound to said human nucleic acid molecules complexed to said proteins thereby producing enriched cell-free microbial nucleic acid molecules; and
(c) generating metagenomic features of said enriched cell-free microbial nucleic acid molecules configured to diagnose a non-oncologic disease.

138. The method of claim 137, wherein said proteins comprise one or more histone proteins, one or more regulatory proteins, or any combination thereof.

139. The method of claim 137, wherein said sample comprises plasma, serum, whole blood, urine, cerebral spinal fluid, saliva, sweat, tears, exhaled breath condensate, or any combination, dilution, or processed fraction thereof.

140. The method of claim 137, wherein said probes comprise one or more antibodies.

141. The method of claim 137, wherein said removing comprises incubating said antibodies bound to said human nucleic acid molecules complexed to proteins with a solid support, wherein said solid support comprises capture reagents configured to bind to said antibodies.

142. The method of claim 137, further comprising contacting said enriched cell-free microbial nucleic acid molecules with a second set of probes, wherein said second set of probes are configured to bind to microbial marker genes.

143. The method of claim 142, wherein said microbial marker genes comprise ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf; or any combination thereof.

144. The method of claim 142, wherein said microbial marker genes are sequenced to determine a taxonomic, functional, or any combination thereof abundance of microbes.

145. The method of claim 137, wherein said sample comprises a liquid biological sample.

146. The method of claim 137, wherein said sample originated from a subject.

147. The method of claim 146, wherein said subject is human or non-human mammal.

148. The method of claim 137, wherein said proteins comprise histone proteins associated with nucleic acid molecules.

149. The method of claim 137, wherein said human nucleic acid molecules comprise DNA, RNA, cell-free RNA, cell-free DNA, exosomal RNA, exosomal DNA, or any combination thereof.

150. The method of claim 137, wherein said cell-free microbial nucleic acid molecules comprise cell-free microbial DNA, cell-free microbial RNA, microbial RNA, microbial DNA, or any combination thereof.

151. The method of claim 137, wherein said removing comprises immunoprecipitating said probes bound to said human nucleic acid molecules.

152. The method of claim 150, further comprising preparing a single stranded library from said cell-free microbial nucleic acid molecules of said sample.

153. The method of claim 137, wherein said probes are coupled to a solid support.

154. The method of claim 153, wherein said solid support comprises a bead, magnetic bead, agarose bead, non-magnetic latex, functionalized Sepharose, pH-sensitive polymers or any combination thereof.

155. The method of claim 137, wherein said sample comprises human nucleic acid molecules, microbial nucleic acid molecules, or any combination thereof.

156. The method of claim 137, wherein said non-oncologic disease comprise benign neoplasms of the integumentary, skeletal, muscular, nervous, endocrine, cardiovascular, lymphatic, digestive, respiratory, urinary, reproductive, or any system combinations thereof.

157. A method of generating a microbial metagenomic feature set to diagnose a non-oncologic disease, the method comprising:
(a) providing a plurality of subjects' health states and said plurality of subjects' biological samples, wherein said biological samples comprise mammalian nucleic acid molecules and microbial nucleic acid molecules;
(b) removing said mammalian nucleic acid molecules from said biological samples with an affinity capture reagent;
(c) sequencing said microbial nucleic acid molecules to generate microbial sequencing reads; and
(d) generating said microbial metagenomic feature set to diagnose a non-oncologic disease by combining a metagenomic feature abundances of said microbial sequencing reads and said plurality of subjects' health states.

158. The method of claim 157, wherein said metagenomic feature set comprises microbial taxonomic abundance.

159. The method of claim 157, wherein said metagenomic feature set comprises computationally inferred microbial biochemical pathways and said microbial biochemical pathways' associated abundances.

160. The method of claim 157, wherein said metagenomic feature set comprises microbial phylogenetic marker genes or marker gene fragments thereof.

161. The method of claim 157, wherein said sample comprises a liquid biological sample, and wherein said liquid biological sample comprises: plasma, serum, whole blood, urine, cerebral spinal fluid, saliva, sweat, tears, exhaled breath condensate, or any combination, dilution, or processed fraction thereof.

162. The method of claim 161, wherein (b) comprises:
(a) contacting said liquid biological sample with a solid support comprising immobilized anti-nucleosome antibodies to form antibody-nucleosome interaction complexes;
(b) separating said solid support from said liquid biological sample to concentrate said antibody-nucleosome interaction complexes; and
(c) purifying the remaining one or more nucleosome-depleted microbial nucleic acid molecules.

163. The method of claim 162, wherein said anti-nucleosome antibodies are configured to bind to an epitope comprising DNA and one or more histone proteins.

164. The method of claim 162, wherein said solid support comprise a magnetic bead, agarose bead, non-magnetic latex, functionalized Sepharose, pH-sensitive polymers or any combination thereof.

165. The method of claim 161, wherein (b) comprises:
(a) contacting said liquid biological sample with anti-nucleosome antibodies to form antibody-nucleosome interaction complexes;
(b) contacting said antibody-nucleosome interaction complexes with a solid support;
(c) separating said solid support from said liquid biological sample to concentrate said antibody-nucleosome interaction complexes; and
(d) purifying the remaining nucleosome-depleted microbial nucleic acid molecules.

166. The method of claim 165, wherein said anti-nucleosome antibodies comprise a plurality of epitope tags.

167. The method of claim 166, wherein said plurality of epitope tags comprise an N- or C-terminal 6×-histidine tag, green fluorescent protein (GFP), myc, hemagglutinin (HA), Fc fusion, biotin or any combination thereof.

168. The method of claim 165, wherein said solid support comprises a magnetic bead, agarose bead, non-magnetic latex, functionalized Sepharose, pH-sensitive polymers or any combination thereof.

169. The method of claim 165, wherein said solid support comprises covalently immobilized affinity agents.

170. The method of claim 169, wherein said affinity reagents comprise streptavidin, antibodies specific for 6×-histidine tag, green fluorescent protein (GFP), myc, hemagglutinin (HA), biotin, or any combination thereof.

171. The method of claim 169, wherein said affinity agents comprise anti-species antibodies.

172. The method of claim 157, wherein (c) comprises:
(a) generating single-stranded DNA libraries from said microbial nucleic acid molecules;
(b) performing shotgun metagenomic sequencing analysis of said single-stranded DNA libraries to produce sequencing reads;
(c) filtering said sequencing reads to produce mammalian DNA-depleted microbial sequencing reads; and
(d) decontaminating said mammalian DNA-depleted microbial sequencing reads to remove non-endogenous microbial sequencing reads.

173. The method of claim 172, wherein said decontaminating comprises in-silico decontamination.

174. The method of claim 172, wherein said filtering comprises computationally mapping said sequencing reads to a human reference genome database.

175. The method of claim 157, wherein (c) comprises:
(a) amplifying genomic features of said microbial nucleic acid molecules, thereby generating amplified genomic features;
(b) sequencing said amplified genomic features to generate sequencing reads;
(c) filtering said sequencing reads to produce mitochondrial DNA-depleted microbial sequencing reads; and
(d) decontaminating said mitochondrial DNA-depleted microbial sequencing reads to remove non-endogenous microbial sequencing reads.

176. The method of claim 175, wherein said decontaminating comprises in-silico decontamination.

177. The method of claim 175, wherein said genomic features comprise microbial phylogenetic marker genes or marker gene fragments thereof.

178. The method of claim 177, wherein said microbial phylogenetic marker genes comprise bacterial marker genes or marker gene fragments thereof.

179. The method of claim 177, wherein said microbial phylogenetic marker genes comprise fungal marker genes or marker gene fragments thereof.

180. The method of claim 178, wherein said bacterial marker genes comprise: ribosomal RNA gene 5S; ribosomal RNA gene 16S; ribosomal RNA gene 23S; bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

181. The method of claim 179, wherein said fungal marker genes comprise one or more of: ribosomal RNA gene 18S, ribosomal RNA gene 5.8S, ribosomal RNA gene 28S, and the internal transcribed spacer regions 1 and 2.

182. The method of claim 177, wherein said microbial phylogenetic marker genes comprise bacterial, fungal, or any combination thereof marker genes.

183. The method of claim 175, wherein amplifying comprises performing a polymerase chain reaction or derivatives thereof.

184. The method of claim 183, wherein said derivatives thereof comprise inverse PCR, anchored PCR, primer-directed rolling circle amplification, or any combination thereof.

185. The method of claim 157, wherein (c) comprises enriching said microbial nucleic acid molecules.

186. The method of claim 185, wherein said enriching comprises:
(a) combining said microbial nucleic acid molecules with hybridization probes, wherein said hybridization probes comprise a nucleic acid sequence complementarity to microbial genomic features;
(b) incubating said hybridization probes and said microbial nucleic acid molecules under conditions that promote nucleic acid base pairing between target nucleic acid features and said hybridization probes;
(c) separating unbound hybridization probes and hybridized probes bound to said microbial nucleic acid molecules; and
(d) washing said hybridized probes bound to said microbial nucleic acid molecules, thereby generating enriched microbial nucleic acid molecules.

187. The method of claim 186, wherein said washing is configured to remove non-specifically associated nucleic acid molecules and other reaction components.

188. The method of claim 185, wherein said enriching comprises:
(a) combining said microbial nucleic acid molecules with recombinant CXXC-domain proteins to form a protein-DNA binding reaction;
(b) incubating said protein-DNA binding reaction under conditions that promote an interaction between said recombinant CXXC-domain proteins and non-methylated CpG motifs of said microbial nucleic acid molecules; and
(c) separating unbound recombinant CXXC-domain proteins and recombinant CXXC-domain proteins bound to said non-methylated CpG motifs from a remainder of said protein-DNA binding reaction; and
(d) washing said recombinant CXXC-domain proteins bound to said non-methylated CpG nucleic acid fragments, thereby generating enriched nucleic acid molecules for amplification.

189. The method of claim 188, wherein said washing is configured to remove non-specifically associated nucleic acid molecules and said remainder of protein-DNA binding reaction components.

190. The method of claim 157, wherein said plurality of subjects comprise human, non-human mammal, or any combination thereof subjects.

191. The method of claim 157, wherein said mammalian nucleic acid molecules comprise DNA, RNA, cell-free DNA, cell-free RNA, exosomal DNA, exosomal RNA, or any combination thereof nucleic acid molecules, and wherein said microbial nucleic acid molecules comprise microbial cell-free RNA, microbial cell-free DNA, microbial RNA, microbial DNA, or any combination thereof nucleic acid molecules.

192. The method of claim 157, further comprising generating a trained predictive model, wherein said trained predictive model is trained with said microbial metagenomic feature set and said health state of said one or more subjects of said plurality of subjects.

193. The method of claim 192, wherein said trained predictive model comprises a machine learning model, one or more machine learning models, an ensemble of machine learning models, or any combination thereof.

194. The method of claim 192, wherein said trained predictive model comprises a regularized machine learning model.

195. The method of claim 193, wherein said machine learning model comprises a machine learning classifier.

196. The method of claim 193, wherein said machine learning model comprises a gradient boosting machine, neural network, support vector machine, k-means, classification trees, random forest, regression, or any combination thereof machine learning models.

197. The method of claim 157, wherein said non-oncologic disease comprises benign neoplasms of the integumentary, skeletal, muscular, nervous, endocrine, cardiovascular, lymphatic, digestive, respiratory, urinary, reproductive, or any system combinations thereof.

198. The method of claim 157, wherein said subjects' health states comprise said subjects' known non-oncologic disease.
